(19) United States
(12) Patent Application Publication
(10) Pub. No.: US 2020/0029946 A1
(43) Pub. Date: Jan. 30, 2020

(54) SUCTION CLOSURE DEVICES AND METHODS

(71) Applicant: Merit Medical Systems, Inc., South Jordan, UT (US)

(72) Inventors: Stephen M. Green, Syracuse, NY (US); Ronald P. Caputo, Manlius, NY (US); John M. Kirwan, Wilbraham, MA (US); Aaron Hopkinson, Herriman, UT (US); Jacquelyn Monroe, Highland, UT (US)

(21) Appl. No.: 16/521,897

(22) Filed: Jul. 25, 2019

Related U.S. Application Data
(60) Provisional application No. 62/703,545, filed on Jul. 26, 2018.

(51) Int. Cl. A61B 17/00; A61B 17/08

(52) U.S. Cl. CPC A61B 17/0057; A61B 17/08; A61B 2017/00606; A61B 2017/00615; A61B 2017/00623; A61B 2017/00628; A61B 2017/00637

(57) ABSTRACT

Devices and methods for creating hemostasis at a subcutaneous vascular puncture are disclosed. The methods and devices may be used to close vascular punctures following trans-radial arterial procedures, e.g., catheterization and percutaneous coronary intervention. The devices may include a housing defining a suction chamber and an anvil disposed within the suction chamber.

### RELATED APPLICATION

This application claims priority to U.S. Provisional Application No. 62/703,545, filed on Jul. 26, 2018 and titled “Suction-Actuated Trans-Radial Closure Device and Method of Operation” which is hereby incorporated by reference in its entirety.

### TECHNICAL FIELD

The present disclosure relates to medical devices, and more particularly, to a vascular puncture hemostasis apparatus following trans-radial arterial procedures.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1A is a top perspective view of an embodiment of an arteriotomy closure device.

FIG. 1B is a bottom perspective view of the embodiment of the arteriotomy closure device of FIG. 1A.

FIG. 2A is a bottom perspective view of an embodiment of a housing of a suction chamber and an anvil disposed therein.

FIG. 2B is a bottom perspective view of an embodiment of a housing of a suction chamber and an anvil disposed therein.

FIG. 3A is a bottom perspective view of an embodiment of a housing of a suction chamber and an anvil disposed therein.

FIG. 3B is a bottom perspective view of an embodiment of a housing of a suction chamber and an anvil disposed therein.

FIG. 4A is a bottom perspective view of an embodiment of a housing of a suction chamber and an anvil disposed therein.

FIG. 4B is a bottom perspective view of an embodiment of a housing of a suction chamber and an anvil disposed therein.

FIG. 5A is a bottom perspective view of an embodiment of a housing of a suction chamber and an anvil disposed therein.

FIG. 5B is a bottom perspective view of an embodiment of a housing of a suction chamber and an anvil disposed therein.

FIG. 6A is a top perspective view of an embodiment of an arteriotomy closure device.

FIG. 6B is a bottom perspective view of an embodiment of the arteriotomy closure device of FIG. 6A.

FIG. 7A is a partially cut-away bottom perspective view of an embodiment of an arteriotomy closure device.

FIG. 7B is a partially cut-away top perspective view of an embodiment of the arteriotomy closure device of FIG. 7A.

FIG. 8A is a partially cut-away bottom perspective view of an embodiment of an arteriotomy closure device.

FIG. 8B is a partially cut-away top perspective view of an embodiment of the arteriotomy closure device of FIG. 8A.

FIG. 9A is a partially cut-away bottom perspective view of an embodiment of an arteriotomy closure device.

FIG. 9B is a partially cut-away top perspective view of an embodiment of the arteriotomy closure device of FIG. 9A.

FIG. 10A is a partially cut-away bottom perspective view of an embodiment of an arteriotomy closure device.

FIG. 10B is a partially cut-away top perspective view of an embodiment of the arteriotomy closure device of FIG. 10A.

FIG. 11A is a partially cut-away bottom perspective view of an embodiment of an arteriotomy closure device.

FIG. 11B is a partially cut-away top perspective view of an embodiment of the arteriotomy closure device of FIG. 11A.

FIG. 12 is a perspective view of an embodiment of an arteriotomy closure device.

FIG. 12A is a bottom perspective view of an embodiment of the arteriotomy closure device of FIG. 12.

FIG. 12B is a top perspective view of an embodiment of the arteriotomy closure device of FIG. 12.

FIG. 12C is a perspective view of an embodiment of the arteriotomy closure device of FIG. 12 disposed on a limb of a patient.

FIG. 13 is a perspective view of an embodiment of an arteriotomy closure device.

FIG. 13A is an exploded perspective view of an embodiment of the arteriotomy closure device of FIG. 13.

FIG. 13B is a top perspective view of an embodiment of the arteriotomy closure device of FIG. 13.

FIG. 13C is a bottom perspective view of an embodiment of the arteriotomy closure device of FIG. 13.

FIG. 13D is a perspective view of an embodiment of the arteriotomy closure device of FIG. 13 disposed on a limb of a patient.

FIG. 14A is a cross-sectional view of an arteriotomy closure device coupled to a limb of a patient without suction.

FIG. 14B is a cross-sectional view of the arteriotomy closure device of FIG. 14A coupled to a patient's limb with suction.

FIG. 15A is a schematic illustration of a longitudinal ultrasound image of a portion of a patient's limb without a suction force applied to a skin surface.

FIG. 15B is a schematic illustration of a longitudinal ultrasound image of a portion of a patient's limb with a suction force applied to a skin surface.

FIG. 16A is a schematic illustration of a transverse cross-sectional view of a portion of a patient's limb without a suction force applied to a skin surface.

FIG. 16B is a schematic illustration of a transverse ultrasound image of a portion of a patient's limb with a suction force applied to a skin surface.

FIG. 17 is a perspective view of an arteriotomy closure system or kit.

### DETAILED DESCRIPTION

Various medical procedures, particularly cardiology procedures, involve accessing a corporeal blood vessel through a percutaneous sheath. Insertion of the sheath necessarily requires an opening, or puncture wound, in the blood vessel so that a medical procedure can be performed through the sheath. After the medical procedure has been completed, the sheath must be removed from the blood vessel and the access hole in the blood vessel must be closed to create cessation of bleeding from the blood vessel.

As an alternative to the historically standard access to the vasculature via the femoral artery in a patient's groin, access via an artery in a patient's wrist (i.e., either the radial artery or the ulnar artery) has gained recent popularity. This is particularly due to lessened post-procedure access site bleeding complications. The standard means for inducing post-procedure hemostasis of either a radial artery or an ulnar artery is to apply direct pressure to the patient's wrist approximate of the subcutaneous sheath entry site, or arteriotomy. Several devices have been introduced into the device market which aid in applying such direct pressure to a patient's wrist. These hemostasis devices are frequently composed of a wrist band with a means for applying direct contact pressure on the patient's inside wrist skin surface approximate to the subcutaneous vessel's puncture wound. In order for such devices to effectively create hemostasis at the artery's puncture site, or arteriotomy, they must necessarily compress the soft tissue overlying the blood vessel. Such wrist band type devices may incorporate an inflatable balloon element for further focusing the direct pressure at a position on the patient's wrist overlying the arterial puncture.

By design, these inflatable balloon compression devices apply pressure to the patient's wrist, spread over a relatively small area with enough inward force to effect cessation of bleeding from the arteriotomy. Such application of pressure to the wrist surface, however, may result, in certain instances, in flattening, or collapsing, of the lumen of the subcutaneous artery. When the arterial lumen collapses, the blood flow path through the artery is narrowed. This can result in arterial occlusion through a variety of mechanisms—particularly when there is complete cessation of arterial flow during compression. Such occlusion of a radial artery and the resulting non-patency can create reduced blood flow to the patient's hand, as well as render the radial artery unusable for future percutaneous procedures. Arterial occlusion occurs in approximately 5%-12% of patients undergoing procedures through the radial artery approach and therefore relates to a substantial patient population, particularly in high volume hospitals.

In addition to the probability of arterial occlusion following application of direct pressure at the access site, there are also patient complaints of access site pain and discomfort owing to the prolonged clamping and direct pressure to the inside wrist surface. The pressure applied to the wrist area by a radial compression band, by design, is diffuse in nature. This can lead to venous congestion of the hand and has been reported by patients as painful.

The pain experienced by patients undergoing radial compression can be unpleasant. The arteriotomy closure device disclosed herein offers a method for facilitating hemostasis at a radial or ulnar artery puncture while avoiding the deleterious conditions that can result including pain, discomfort and arterial occlusion during administration of direct pressure at the access site.

The arteriotomy closure device disclosed herein may use a suction force applied to the skin surface to create extension of the skin, the underlying connective tissues, and the artery. In short, a disclosed method involves pulling the artery via its surrounding soft tissue against a compression member or anvil.

As determined empirically via direct ultrasound visualization, under the application of suction to the skin surface, the underlying artery is drawn in an upward direction while maintaining the natural cylindrical cross-sectional geometry of the artery's lumen without collapsing the lumen and resultantly choking the flow of blood. By drawing the artery and its surrounding subcutaneous connective tissue in a direction closer to the skin (and thereby compressing the soft subcutaneous tissue overlying the artery), it is then possible to apply a more concentrated pressure point (or line of pressure) directed specifically at the arteriotomy and with a significantly lower net inward force, thus avoiding overall collapse of the arterial lumen. It is further an assertion of this method that it is considerably more comfortable for the patient than the direct compression technique.

One embodiment of a closure device includes a suction chamber which is placed in contact with the patient's wrist directly overlying the arterial puncture site. The suction chamber may include a sealing surface on its distal end which is placed in direct contact with the patient's skin. The sealing surface may be configured to provide a smooth surface with which to seal to the skin surface such that when a negative gauge pressure or suction force is applied to the suction chamber, the sealing surface will not leak. The addition of a low modulus/low durometer material (e.g., silicone or thermoplastic elastomer) gasket may be incorporated onto the sealing surface to ensure that the appropriate suction force is maintained.

Further, the suction chamber may be fixed to a securement band or wristband. The wristband may have a hook and loop closure or other mechanism for adjustment on the patient's wrist such that when a suction force or negative gauge pressure is applied to the suction chamber, the wrist band supports the suction chamber, allowing maintenance of continuous suction while providing counter pressure at the back side of the patient's wrist or limb.

The suction chamber may also include a stationary, distally protruding counter force member that provides and maintains a localized counter force against the patient's skin (directly overlying and/or approximate to the arteriotomy) during which time a suction force or negative gauge pressure is applied to the suction chamber. The patient's skin is drawn into the distal end of the device (i.e., puckered in an outward direction) and is approximated against the protruding anvil. Along with the skin being distended into the suction chamber, so too is the underlying tissue (including the artery) being drawn in an outward, or distended, position. While the suction force is being applied to the suction chamber, the stationary protruding counter force member exerts a localized counter force (downward force) against the skin, the underlying tissue, and the artery wall, to stop the flow of blood from the arteriotomy; without collapsing the arterial lumen which could create thrombosis and occlusion of the artery.

The stationary protruding counter force member or anvil may be implemented in one of multiple configurations. The counter force member can be a single flat surface, a rib configured as a longitudinal keel, a cross-pattern, a cylindrical boss, or a series of concentric bearing surfaces, for example. The distal margin of the counter force member which contacts the skin and compresses the tissues can be shaped to be flat or convex, for example.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use. As specifically applied to the housing of an arteriotomy closure device, the proximal end of the housing refers to the top of the housing which is away from the patient's skin and the distal end refers to the opposite end, the end nearest the patient's skin when the arteriotomy closure device is in use. Thus, if at one or more points in a procedure a physician changes the orientation of the housing, as used herein, the term “proximal end” always refers to the top end of the housing (even if the distal end is temporarily closer to the physician).

“Fluid” is used in its broadest sense, to refer to any fluid, including both liquids and gases as well as solutions, compounds, suspensions, etc., which generally behave as fluids.

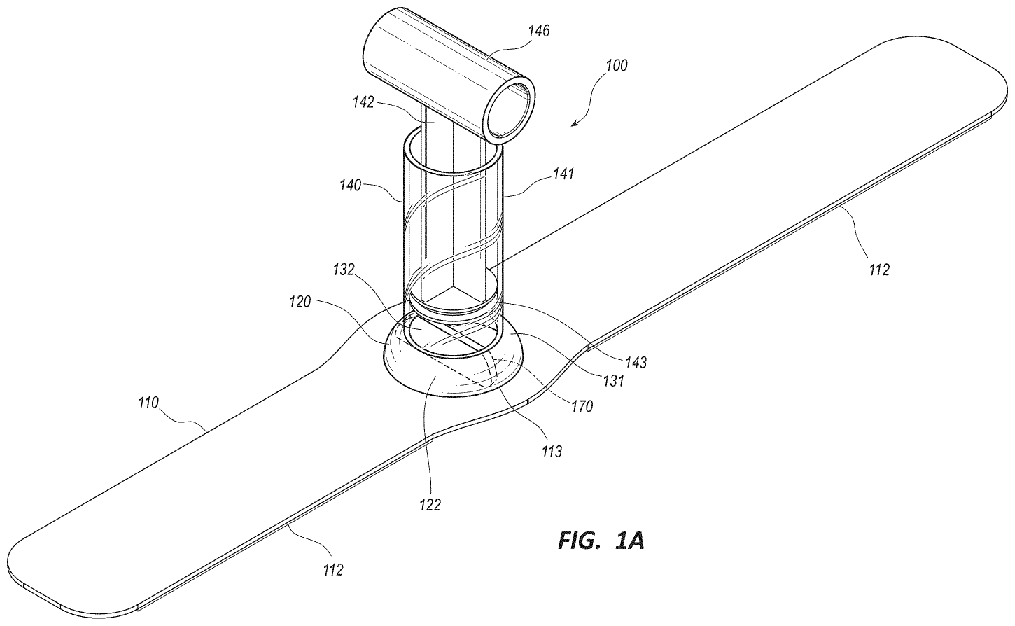

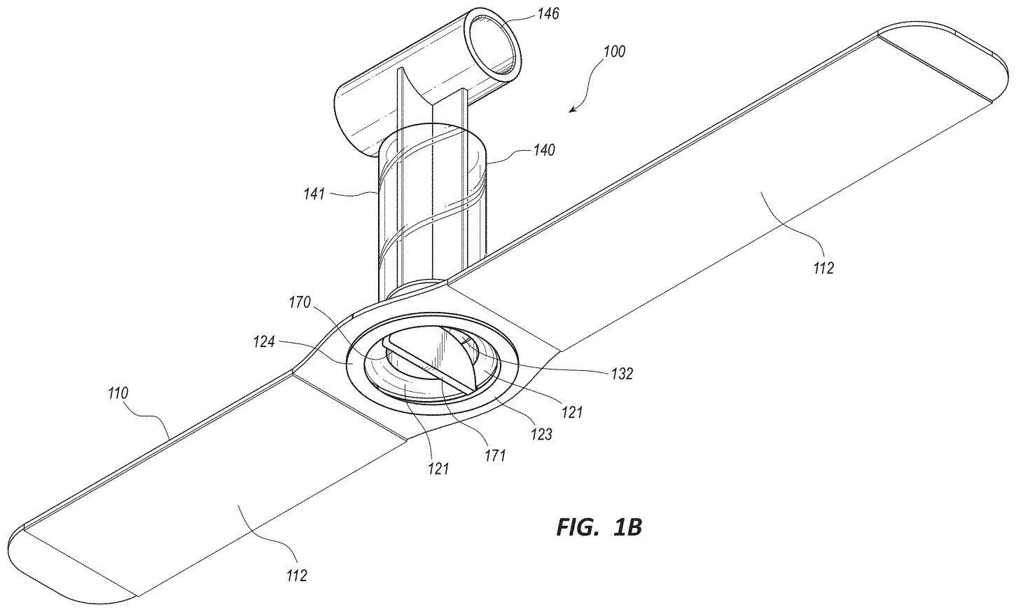

FIGS. 1A-14 illustrate different views of several arteriotomy closure devices and related components. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

FIGS. 1A-1B show an embodiment of an arteriotomy closure device 100. The arteriotomy closure device 100 includes a housing 120, a counter force member or anvil 170, a fluid displacement or suction member 140, and a securement band 110. The housing 120 may have a generally elliptical shape and may be formed from a material that is sufficiently flexible to form a seal at a treatment site while being sufficiently rigid to withstand negative gauge pressure applied within the housing and not collapse. For example, in one embodiment, the housing 120 may be formed from a flexible material, such as silicone, polyurethane (e.g., Tecoflex, Chronoflex, Carbothane or other aliphatic or aromatic polyurethanes), thermoplastic elastomer, etc., such that the housing 120 is conformable to a contour of an anterior aspect of a patient's wrist or limb. In another embodiment, the housing 120 may be formed from a rigid or semi-rigid material, such as polycarbonate, high density polyurethane, polypropylene, etc., such that the housing is formed with a contour shape configured to conform to the anterior aspect of the patient's wrist. In one embodiment the durometer of the material is between 70 and 100 on the Shore A scale. In another embodiment, the durometer of the material is between 80 and 90 on the Shore A scale. The housing 120 can be formed using any suitable manufacturing technique, such as injection molding, casting, 3-D printing, etc. The housing may be transparent or translucent to allow a user to view the inside of the housing 120.

The housing 120, as illustrated in the depicted embodiment of FIGS. 1A-1B, includes a side wall 122 extending distally from a top surface 131. The side wall 122 terminates at a distal sealing surface 123. The housing 120 and/or the distal sealing surface 123 may consist of arcuate features that avoid straight lines or sharp corners to effectively hold negative gauge pressure. The distal sealing surface 123 may include a flange 124 extending radially outward from the distal sealing surface 123. The flange 124 may be configured to increase a surface area of the sealing surface 123 to enhance sealing of the housing against the skin of a patient. In other embodiments, the distal sealing surface 123 may include a smooth, flat surface with a width that is equivalent to a thickness of the side wall 122. In another embodiment, a compressible gasket may be coupled to the sealing surface 123 to enhance the sealability of the sealing surface 123 to the patient's skin. The gasket may be formed of any suitable low modulus, low durometer material, such as silicone, polyurethane, thermoplastic elastomer, closed cell foam, etc. The gasket can be coupled to the sealing surface 123 using any suitable manufacturing technique, such as over molding, gluing, bonding, etc. In some embodiments, a topical gel may be applied to the patient's skin to achieve and maintain a leak-proof seal between the distal sealing surface 123 and the patient's skin. In certain embodiments, the distal sealing surface 123 can include a suction cup or a plurality of suction cups to promote stabilization of the housing 120. The side wall 122 and the top surface 131 of the housing 120 can define a suction chamber 121.

The anvil 170 is shown in the illustrated embodiment of FIG. 1B disposed within the suction chamber 121 and extending distally from the top surface 131. The anvil 170 can divide the suction chamber 170 into at least two portions. The anvil 170 includes a distal end 171 which is recessed into the suction chamber 121. In other embodiments, the distal end 171 may be flush with the sealing surface 123. The anvil 170 may be integral with the housing 120 and formed from the same material as the housing 120. In other embodiments, the anvil 170 may be a separate component of a different material and coupled to the housing using any suitable technique, such as over molding, gluing, bonding, etc. In certain embodiments, the anvil 170 may be flexible. In other embodiments, the anvil 170 may be rigid or semi-rigid.

The anvil 170 may be of any suitable shape such that a patient's skin, subcutaneous tissue, and artery can be drawn against the distal end 171 of the anvil 170 to close an arteriotomy. FIGS. 2A-5B depict exemplary shapes of the anvil 170. Any one of the exemplary anvil shapes may be used with any one of the arteriotomy closure embodiments disclosed herein.

Turning to FIG. 2A, a bottom perspective view of a housing 120a is shown. FIG. 2A illustrates an anvil 170a configured as a cross pattern. A distal end 171a of the anvil 170a may include a coating of indicia such that the indicia (e.g., a contrasting colored cross) aid the operator in aligning the housing 120a and the anvil 170a to be centered directly over the arteriotomy.

Turning to FIG. 2B, a bottom perspective view of a housing 120b is shown. FIG. 2B illustrates an anvil 170b configured as a cross pattern with a substantially curved, or convex, distal end 171b. The distal end 171b may include a coating of indicia such that the indicia aid the operator in aligning the housing 120b and the anvil 170b to be centered directly over an arteriotomy.

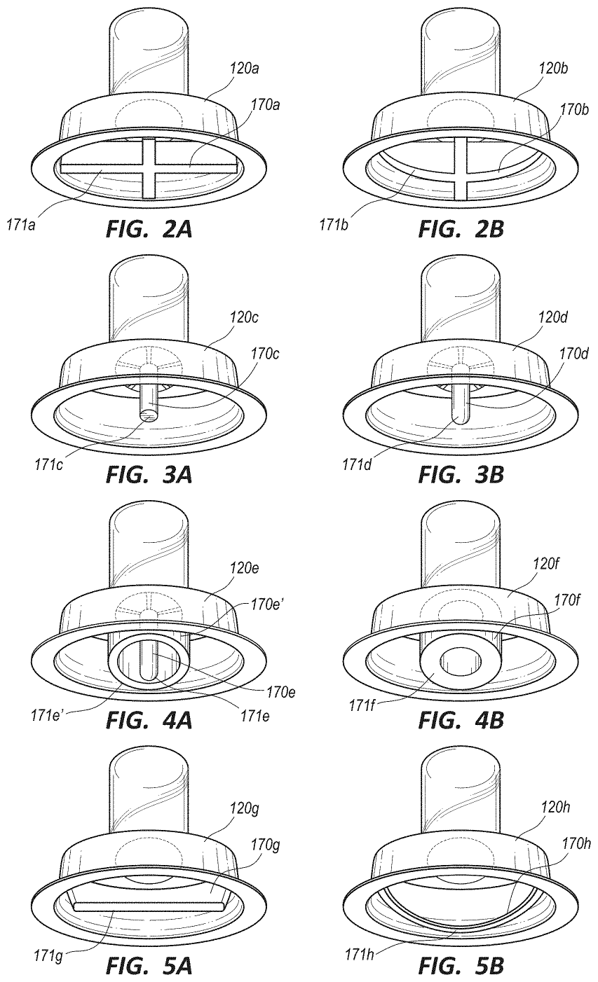

Turning to FIG. 3A, a bottom perspective view of a housing 120c is shown. FIG. 3A illustrates an anvil 170 configured as a central cylindrical boss with a flat distal end 171c. The distal end 171c may include a coating of indicia such that the indicia aid the operator in aligning the housing 120c and the anvil 170c to be centered directly over the arteriotomy.

Turning to FIG. 3B, a bottom perspective view of a housing 120d is shown. FIG. 3B illustrates a central cylindrical anvil 170d with a substantially smooth radiused distal end 171d. The distal end 171d may include a coating of indicia such that the indicia aid the operator in aligning the housing 120d and the anvil 170d to be centered directly over the arteriotomy.

Turning to FIG. 4A, a bottom perspective view of a housing 120e is shown. FIG. 4A illustrates a configuration of a central cylindrical anvil 170e with a substantially smooth radiused distal end 171e. A concentric outer cylindrical anvil 170e′ surrounds the central anvil 170e. Both the distal end 171e of the central cylindrical anvil 170e and the distal end 171e′ of the concentric outer cylindrical anvil 170e′ are configured such that they extend the same distance in the distal direction (i.e., the distal ends 171e, 171e′ of both anvils 170e, 170e′ are coplanar); however, because of the geometry of the distended skin under the application of suction being applied to the housing 120e such that a seal is formed at the interface between the housing 120e and the patient's skin, the distal end 171e of the central anvil 170e comes into contact with the patient's skin before the distal end 171e′ of the outer anvil 170e′ comes into contact with the patient's skin and thereby, the contact pressure exerted on the skin by the distal end 171e of the central anvil 170e is greater in magnitude than the contact pressure exerted on the skin by the distal end 171e′ of the outer mandrel 170e′. Distal ends 171e, 171e′ of the anvils 170e, 170e′ may include a coating of indicia such that the indicia (e.g., a contrasting colored bullseye) aid the operator in aligning the housing 120e and the anvils 170e, 170e′ to be centered directly over an arteriotomy.

Turning to FIG. 4B, a bottom perspective view of a housing 120f is illustrated. FIG. 4B shows a cylindrical anvil 170f with a substantially flat distal end 171f. A distal end 171f of the anvil 170f may also include a coating of indicia such that the indicia aid the operator in aligning the housing 120f and the anvil 170f to be centered directly over an arteriotomy.

Turning to FIG. 5A, a bottom perspective view of a housing 120g is shown. FIG. 5A illustrates an anvil 170g configured as a central longitudinal rib, or keel. A distal end 171g of the anvil 170g may include a coating of indicia such that the indicia aid the operator in aligning the housing 120g and the anvil 170g to be centered directly over an arteriotomy.

Turning to FIG. 5B, a bottom perspective view of a housing 120h is shown. FIG. 5B illustrates an anvil 170h configured as a central longitudinal rib, or keel having a curved or convex distal end 171h. The distal end 171h may include a coating of indicia such that the indicia aid the operator in aligning the housing 120h and the anvil 170h to be centered directly over an arteriotomy.

Referring again to FIGS. 1A-1B, the illustrated embodiment of the arteriotomy closure device 100 shows the fluid displacement or suction member 140 extending upwardly from the housing 120. The suction member 140 can include a barrel 141 and a plunger 142. The barrel 141 can be integrally formed with the housing 120 and be formed from the same material as the housing 120. In other embodiments, the barrel 141 can be a separate component and be coupled to the housing 120 using any suitable technique, such as bonding, gluing, welding, friction fit, etc. In this embodiment, the barrel 141 may be formed from the same or different material than the housing 120. The housing 120 may include a port 132 disposed between the suction chamber 121 and the barrel 141 such that the suction chamber 121 is in fluid communication with the barrel 141.

The plunger 142 is disposed within the barrel 141 and is configured to be longitudinally displaced from a distal position to a proximal position. The plunger 142 may include a plunger tip 143 coupled to a distal end of the plunger 142. The plunger tip 143 may be configured to seal against an internal surface of the barrel 141. A plunger grip 146 may be disposed at a proximal end of the plunger 142. The plunger grip 146 may be of any suitable shape that allows a user to easily grip the plunger 142 for longitudinal displacement. For example, as illustrated in FIGS. 1A-1B, the plunger grip 146 can be in the form of a transversely oriented cylinder. In other embodiments, the plunger grip 146 may be in the form of a transverse flange, a bulb, a ring, etc. In certain embodiments, the plunger 142 may be held in the proximal position via a plunger retention member in order to maintain continuous vacuum in the suction chamber 121. For example, the plunger retention mechanism may be a ratchet mechanism or detent-type holding mechanism or any other suitable mechanism configured to hold the plunger 142 in a longitudinal position.

As shown in FIGS. 1A-1B, the housing 120 may be coupled to a securement band 110. The securement band 110 may be configured to be disposed around a portion of a patient's limb in order to secure the housing 120 over an arteriotomy site. For example, the securement band 110 may be a wrist band configured to be disposed around a wrist of a patient such that the housing 120 can be secured over a radial, ulnar or palmar artery arteriotomy site. In other embodiments, the securement band 110 may be configured to be disposed around a patient's hand, thigh, ankle, upper arm, etc. In some embodiments the palmar artery may be accessed at the anatomical snuffbox of the patient. The securement band 110 may be formed of a flexible polymeric film and include a fixation member 112 configured to selectively couple ends of the securement band 110 together when disposed around the portion of the patient's limb. For example, the fixation member 112 may be a hook and loop material, buckle, snap, button, adhesive, etc. The securement band 110 may include an aperture 113 through which the housing 120 can be disposed and coupled to the securement band 110.

In use, following an arterial catheterization procedure and prior to removal of an introducer sheath, the arteriotomy closure device 100 may be positioned on a portion of a limb of a patient such that the housing 120 is disposed over an arteriotomy site. In certain embodiments, the housing 120 may be disposed over the arteriotomy site, a skin puncture site, and/or a skin tract between the arteriotomy site and the skin puncture site. The securement band 110 may be wrapped around the portion of the limb with the ends of the securement band 110 releasably fixed together. The housing 120 may be held tightly to the patient's skin such that the distal sealing surface 123 forms an airtight seal between the housing 120 and the patient's skin. In some embodiments, a liquid, gel, or skin protective film may be applied to the patient's skin prior to facilitate achieving the airtight seal. The plunger 142 can be displaced from the distal position to the proximal position to generate a suction within the barrel 141 and the suction chamber 121. The plunger 142 may be locked in the proximal position by the plunger retention member. The suction within the suction chamber 121 may cause the patient's skin, subcutaneous tissue, and artery to be drawn, or distended, in a proximal, or puckered, position toward the housing 120 and anvil 170, such that the anvil 170 applies a counter force against the patient's skin overlying the arteriotomy and/or a tissue tract which facilitates hemostasis, or cessation of bleeding, from the arteriotomy. In certain embodiments, a suction force is formed or induced (i.e., negative gauge pressure is induced) in the suction chamber 121 prior to removal of the introducer sheath from the artery. In other embodiments, the introducer sheath is removed from the artery following the forming of suction in the suction chamber 121 or simultaneously as negative gauge pressure is induced.

When cessation of bleeding from the skin puncture site has been achieved, the plunger 142 may be moved to the distal position such that the suction in the suction chamber 121 is relieved and atmospheric pressure inside the suction chamber 121 is restored, thus removing the counter force provided by the anvil 170 against the patient's skin. At this time, the arteriotomy closure device 100 may be removed from the patient's limb.

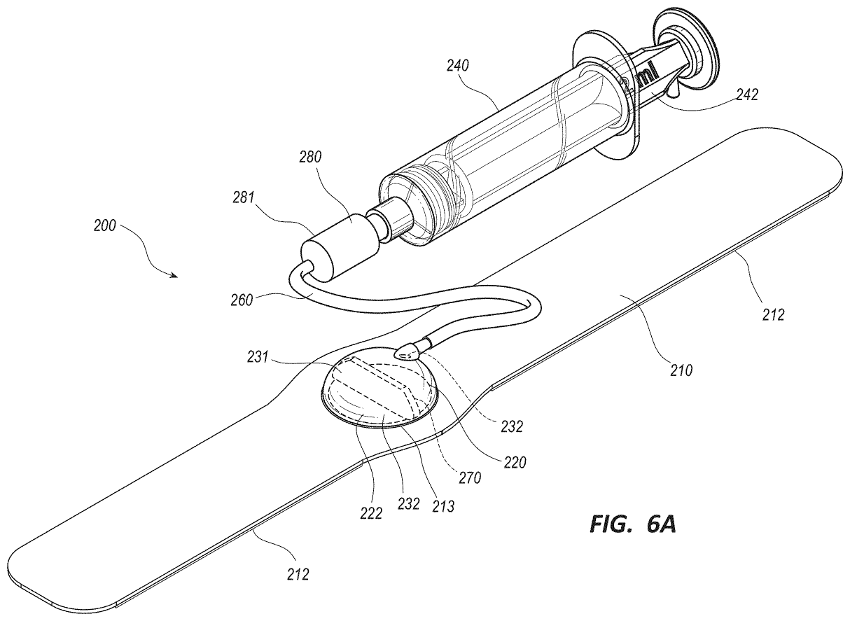

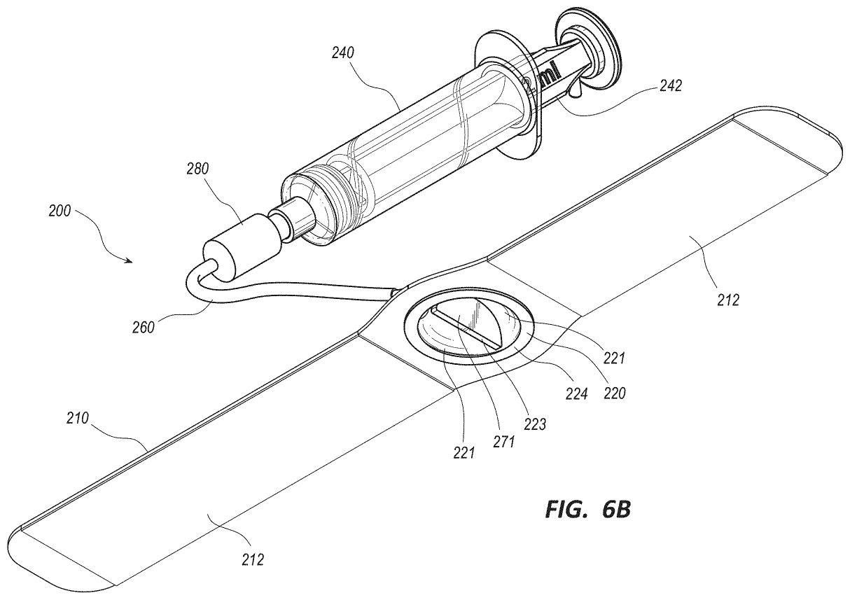

FIGS. 6A-6B depict an embodiment of an arteriotomy closure device 200 that resembles the arteriotomy closure device 100 described above in certain respects. Accordingly, like features are designated with like reference numerals, with the leading digit incremented to “2.” For example, the embodiment depicted in FIGS. 6A-6B includes a housing 220 that may, in some respects, resemble the housing 120 of FIG. 1A. Relevant disclosure set forth above regarding similarly identified features thus may not be repeated hereafter. Moreover, specific features of the arteriotomy closure device 100 and related components shown in FIGS. 1A-1B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the arteriotomy closure device 200 and related components depicted in FIGS. 6A-6B. Any suitable combination of the features, and variations of the same, described with respect to the arteriotomy closure device 100 and related components illustrated in FIGS. 1A-1B, can be employed with the arteriotomy closure device 200 and related components of FIGS. 6A-6B, and vice versa. This pattern of disclosure applies equally to further embodiments depicted in subsequent figures and described hereafter, wherein the leading digits may be further incremented

FIGS. 6A-6B illustrate an embodiment of an arteriotomy closure device 200. The arteriotomy closure device 200 includes a housing 220, a counter force member or anvil 270, an extension tubing 260, a valve member 280, and a securement band 210. The housing 220 may have a generally elliptical shape and may be formed from any suitable material. For example, in one embodiment, the housing 220 may be formed from a flexible material, such as silicone, polyurethane, thermoplastic elastomer, etc., such that the housing 220 is conformable to a contour of an anterior aspect of a patient's limb (e.g., wrist). In another embodiment, the housing 220 may be formed from a rigid or semi-rigid material, such as polycarbonate, high density polyurethane, polypropylene, etc., such that the housing is formed with a contour shape configured to conform to the anterior aspect of the patient's wrist. The housing 220 can be formed using any suitable manufacturing technique, such as injection molding, casting, 3-D printing, etc. The housing may be transparent or translucent to allow a user to view the inside of the housing 220.

The housing 220, as illustrated in the depicted embodiment of FIGS. 6A-6B, includes a side wall 222 extending distally from a top surface 231. The side wall 222 terminates at a distal sealing surface 223. The distal sealing surface 223 may include a flange 224 extending radially outward from the distal sealing surface 223. The flange 224 may be configured to increase a surface area of the sealing surface 223 to enhance sealing of the housing 220 against the skin of a patient. In other embodiments, the distal sealing surface 223 may include a smooth, flat surface with a width that is approximately equivalent to a thickness of the side wall 222. In another embodiment, a compressible gasket may be coupled to the sealing surface 223 to enhance the sealability of the sealing surface 223 to the patient's skin. The gasket may be formed of any suitable low modulus, low durometer material, such as silicone, polyurethane, thermoplastic elastomer, closed cell foam, etc. The gasket can be coupled to the sealing surface 223 using any suitable manufacturing technique, such as over molding, gluing, bonding, etc. In some embodiments, a topical gel, liquid, or skin protective film may be applied to the patient's skin to achieve and maintain a leak-proof seal between the distal sealing surface 223 and the patient's skin. The side wall 222 and the top surface 231 of the housing 220 can define a suction chamber 221 along with the surface of the patient when applied to a treatment site.

The counter force member or anvil 270 is shown in the illustrated embodiment of FIG. 6B to be disposed within the suction chamber 221 and to extend distally from the top surface 231. The anvil 270 can divide the suction chamber 221 into at least two portions. The anvil 270 includes a distal end 271 which is recessed into the suction chamber 221. In other embodiments, the distal end 271 may be flush with the sealing surface 223. The anvil 270 may be integral with the housing 220 and formed from the same material as the housing 220. In other embodiments, the anvil 270 may be a separate component of a different material and coupled to the housing using any suitable technique, such as over molding, gluing, bonding, etc. In certain embodiments, the anvil 270 may be flexible. In other embodiments, the anvil 270 may be rigid or semi-rigid.

The anvil 270 may be of any suitable shape such that a patient's skin, subcutaneous tissue, and artery can be drawn against the distal end 271 of the anvil 270 to close an arteriotomy. FIGS. 2A-5B depict exemplary shapes of the anvil 270. Any one of the exemplary anvil shapes may be used with the arteriotomy closure device 200.

The housing 220, in the illustrated embodiment of FIGS. 6A-6B, includes a port 232 extending radially outwardly from the housing 220. The port 232 is in fluid communication with the suction chamber 221. A distal end of an extension tube 260 is coupled to the port 232. A suction retention member may be coupled to the extension tube. In one embodiment the suction retention member comprises a valve member 280 coupled to the extension tube 260. The valve member 280 may be configured to retain a suction force or negative gauge pressure within the suction chamber 221. In the illustrated embodiment of FIGS. 6A-6B, the valve member 280 is a check-valve 281. The check-valve 281 is coupled to a proximal end of the extension tube 260 such that the check-valve is in fluid communication with the suction chamber 221. The check-valve 281 may be selectively opened to allow a negative gauge pressure or suction force to be induced or formed in the suction chamber 221 and selectively closed to retain the negative gauge pressure or suction force in the suction chamber 221. In other embodiments, the valve member 280 may be of any suitable type capable of selectively opening and closing. For example, the valve member may be a pinch clamp, a slide clamp, a pinch valve, etc.

As shown in FIGS. 6A-6B, the housing 220 may be coupled to a securement band 210. The securement band 210 may be configured to be disposed around a portion of a patient's limb in order to secure the housing 220 over an arteriotomy site. For example, the securement band 210 may be a wrist band configured to be disposed around a wrist of a patient such that the housing 220 can be secured over a radial or ulnar artery arteriotomy site. In other embodiments, the securement band 210 may be configured to be disposed around a patient's hand, thigh, ankle, upper arm, etc. The securement band 210 may be formed of a flexible polymeric film and include a fixation member 212 configured to selectively couple ends of the securement band 210 together when disposed around the portion of the patient's limb. For example, the fixation member 212 may be a hook and loop material, buckle, snap, button, adhesive, etc. The securement band 210 may include an aperture 213 sized to accept the housing 220 such that the housing 220 can be disposed through the aperture 213 and coupled to the securement band 210.

In use, following an arterial catheterization procedure and prior to removal of an introducer sheath, the arteriotomy closure device 200 may be positioned on a portion of a limb of a patient such that the housing 220 is disposed over an arteriotomy site. In certain embodiments, the housing 220 may be disposed over the arteriotomy site, a skin puncture site, and/or a skin tract between the arteriotomy site and the skin puncture site. The securement band 210 may be wrapped around the portion of the limb with the ends of the securement band 210 releasably fixed together. The housing 220 may be held tightly to the patient's skin such that the distal sealing surface 223 forms an airtight seal between the housing 220 and the patient's skin. In some embodiments, a liquid, gel, or skin protective film may be applied to the patient's skin prior to facilitate achieving the airtight seal. A fluid displacement or suction generating member 240 (e.g., syringe) may be coupled to the check-valve 281. A plunger 242 of the syringe 240 can be displaced from the distal position to the proximal position to generate a suction force or negative gauge pressure within the syringe 240 and the suction chamber 221. The plunger 242 may be locked in the proximal position by a plunger retention member. The suction force within the suction chamber 121 may cause the patient's skin, subcutaneous tissue, and artery to be drawn, or distended, in a proximal, or puckered, position toward the housing 220 and anvil 270, such that the anvil 270 applies a counter force against the patient's skin overlying the arteriotomy and/or the tissue tract which facilitates hemostasis, or cessation of bleeding, from the arteriotomy. In certain embodiments, the suction force is formed in the suction chamber 221 prior to removal of the introducer sheath from the artery. In other embodiments, the introducer sheath is removed from the artery following the forming of suction force in the suction chamber 221.

When cessation of bleeding from the skin puncture site has been achieved, the plunger 242 may be moved to the distal position such that the suction force in the suction chamber 221 is relieved and atmospheric pressure inside the suction chamber 221 is restored, thus removing the counter force provided by the anvil 270 against the patient's skin. At this time, the arteriotomy closure device 200 may be removed from the patient's limb.

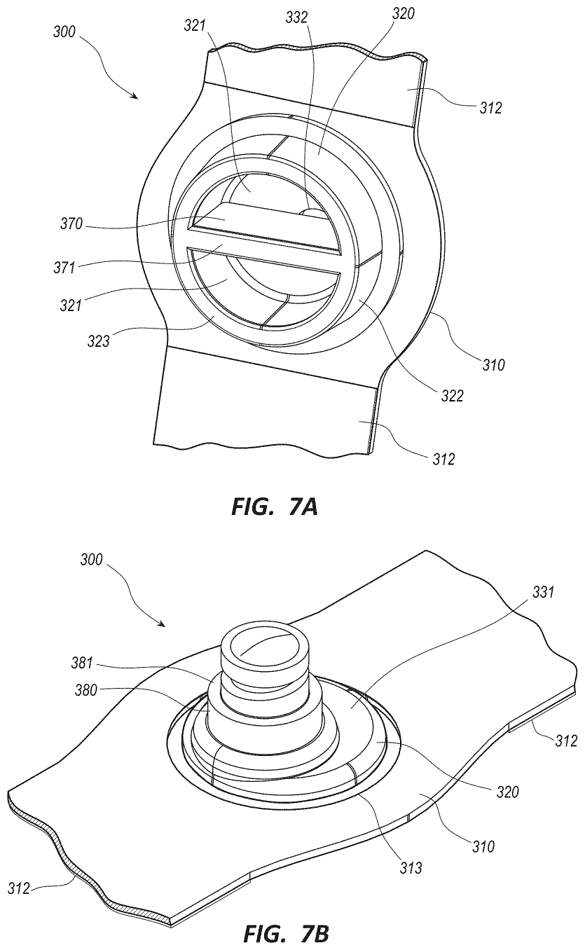

FIGS. 7A-7B illustrate an embodiment of an arteriotomy closure device 300. The arteriotomy closure device 300 includes a housing 320, a counter force member or anvil 370, a valve member 380, and a securement band 310. The housing 320 may have a generally circular shape and may be formed from any suitable material. For example, in one embodiment, the housing 320 may be formed from a flexible material, such as silicone, polyurethane, thermoplastic elastomer, etc., such that the housing 320 is conformable to a contour of an anterior aspect of a patient's limb (e.g., wrist). In another embodiment, the housing 320 may be formed from a rigid or semi-rigid material, such as polycarbonate, high density polyurethane, polypropylene, etc., such that the housing is formed with a contour shape configured to conform to the anterior aspect of the patient's wrist. The housing 320 can be formed using any suitable manufacturing technique, such as injection molding, casting, 3-D printing, etc. The housing may be transparent or translucent to allow a user to view the inside of the housing 320.

The housing 320, as illustrated in the depicted embodiment of FIGS. 7A-7B, includes a side wall 322 extending distally from a top surface 331. The side wall 322 terminates at a distal sealing surface 323. The distal sealing surface 323 may include a smooth, flat surface with a width that is approximately equivalent to a thickness of the side wall 322. In other embodiments, the distal sealing surface 323 may include a flange extending radially outward from the distal sealing surface 323. The flange may be configured to increase a surface area of the sealing surface to enhance sealing of the housing 320 against the skin of a patient. In another embodiment, a compressible gasket may be coupled to the sealing surface 323 to enhance the sealability of the sealing surface 323 to the patient's skin. The gasket may be formed of any suitable low modulus, low durometer material, such as silicone, polyurethane, thermoplastic elastomer, closed cell foam, etc. The gasket can be coupled to the sealing surface 323 using any suitable manufacturing technique, such as over molding, gluing, bonding, etc. In some embodiments, a topical gel, liquid, or skin protective film may be applied to the patient's skin to achieve and maintain a leak-proof seal between the distal sealing surface 323 and the patient's skin. The side wall 322 and the top surface 331 of the housing 320 can define a suction chamber 321 along with the surface of the patient when applied to a treatment site.

The counter force member or anvil 370 is shown in the illustrated embodiment of FIGS. 7A-7B to be disposed within the suction chamber 321 and to extend distally from the top surface 331. The anvil 370 can divide the suction chamber 321 into at least two portions. The two portions in this and other embodiments described herein may still be in fluid communication with each other for even application of suction. The anvil 370 includes a distal end 371 which may be flush with the sealing surface 323. In other embodiments, the distal end 371 may be recessed into the suction chamber 321. The anvil 370 may be integral with the housing 320 and formed from the same material as the housing 320. In other embodiments, the anvil 370 may be a separate component of a different material and coupled to the housing using any suitable technique, such as over molding, gluing, bonding, etc. In certain embodiments, the anvil 370 may be flexible. In other embodiments, the anvil 370 may be rigid or semi-rigid. In yet other embodiments, the anvil 370 may be inflatable.

The anvil 370 of the embodiment illustrated in FIG. 7B extends across a diameter of the housing 320. The distal end 371 is flat and smooth. In other embodiments, the anvil 370 may be of any suitable shape such that a patient's skin, subcutaneous tissue, and artery can be drawn against the distal end 371 of the anvil 370 to close an arteriotomy. FIGS. 2A-5B depict exemplary shapes of the anvil 370. Any one of the exemplary anvil shapes may be used with the arteriotomy closure device 300.

The housing 320, in the illustrated embodiment of FIGS. 7A-7B, includes a port 332 disposed through the top surface 331. The port 332 is in fluid communication with both portions of the suction chamber 321. The port 332 may be disposed off-center from a center point of the top surface 331 in order to allow a user to align the housing 320 with the arteriotomy site. A valve member 380 may be coupled to the top surface 331 such that the valve member 380 is in fluid communication with the suction chamber 321 through the port 332. The valve member 380 may be configured to retain a negative gauge pressure or suction force within the suction chamber 321. In the illustrated embodiment of FIGS. 7A-7B, the valve member 380 is a check-valve 381. The check-valve 381 may be selectively opened to allow a negative gauge pressure or suction force to be formed in the suction chamber 321 and selectively closed to retain the suction force in the suction chamber 321.

As shown in FIGS. 7A-7B, the housing 320 may be coupled to a securement band 310. The securement band 310 may be configured to be disposed around a portion of a patient's limb in order to secure the housing 320 over an arteriotomy site. For example, the securement band 310 may be a wrist band configured to be disposed around a wrist of a patient such that the housing 320 can be secured over a radial or ulnar artery arteriotomy site. In other embodiments, the securement band 310 may be configured to be disposed around a patient's hand, thigh, ankle, upper arm, etc. The securement band 310 may be formed of a flexible polymeric film and include a fixation member 312 configured to selectively couple ends of the securement band 310 together when disposed around the portion of the patient's limb. For example, the fixation member 312 may be a hook and loop material, buckle, snap, button, adhesive, etc. The securement band 310 may include an aperture 313 sized to accept the housing 320 such that the housing 320 can be disposed through the aperture 313 and coupled to the securement band 310.

In use, following an arterial catheterization procedure and prior to removal of an introducer sheath, the arteriotomy closure device 300 may be positioned on a portion of a limb of a patient such that the housing 320 is disposed over an arteriotomy site. In certain embodiments, the housing 320 may be disposed over the arteriotomy site, a skin puncture site, and/or a skin tract between the arteriotomy site and the skin puncture site. The securement band 310 may be wrapped around the portion of the limb with the ends of the securement band 310 releasably fixed together. The housing 320 may be held tightly to the patient's skin such that the distal sealing surface 323 forms an airtight seal between the housing 320 and the patient's skin. In some embodiments, a liquid, gel, or skin protective film may be applied to the patient's skin prior to placement of the housing 320 to facilitate achieving the airtight seal. A fluid displacement suction generating member (e.g., syringe) may be coupled to the check-valve 381. A plunger of the syringe can be displaced from a distal position to a proximal position to generate a suction force or negative gauge pressure within the syringe and the suction chamber 321. The plunger may be locked in the proximal position by a plunger retention member. The suction force or negative gauge pressure within the suction chamber 321 may cause the patient's skin, subcutaneous tissue, and artery to be drawn, or distended, in a proximal, or puckered, position toward the housing 320 and anvil 370, such that the anvil 370 applies a counter force against the patient's skin overlying the arteriotomy and/or the tissue tract which facilitates hemostasis, or cessation of bleeding, from the arteriotomy. In certain embodiments, the suction force is formed in the suction chamber 321 prior to removal of the introducer sheath from the artery. In other embodiments, the introducer sheath is removed from the artery following forming of the suction force in the suction chamber 321.

When cessation of bleeding from the skin puncture site has been achieved, the plunger 342 may be moved to the distal position such that the suction force in the suction chamber 321 is relieved and atmospheric pressure inside the suction chamber 321 is restored, thus removing the counter force provided by the anvil 370 against the patient's skin. At this time, the arteriotomy closure device 300 may be removed from the patient's limb.

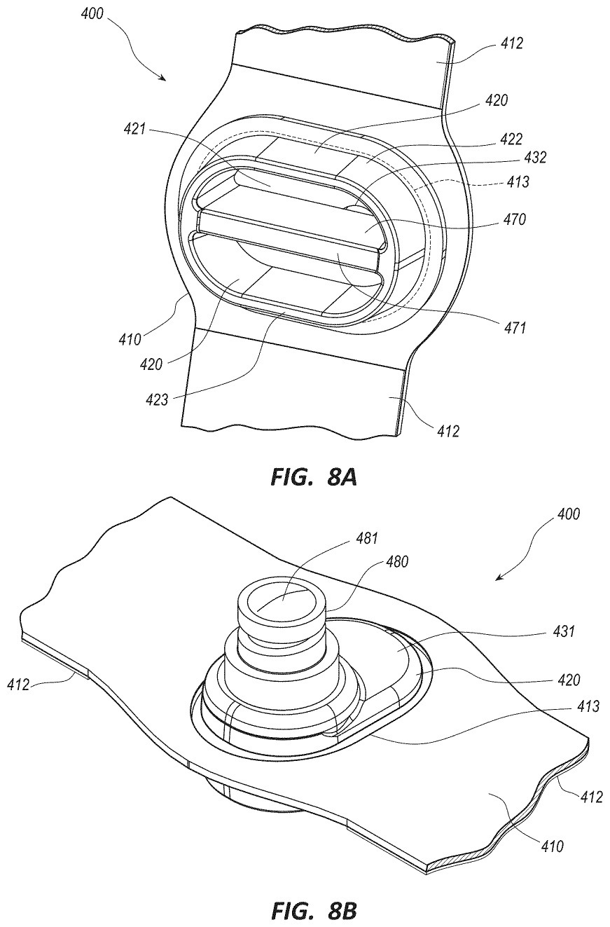

FIGS. 8A-8B illustrate an embodiment of an arteriotomy closure device 400. The arteriotomy closure device 400 includes a housing 420, a counter force member or anvil 470, a valve member 480, and a securement band 410. The housing 420 may have a generally oval or racetrack shape and may be formed from any suitable material. For example, in one embodiment, the housing 420 may be formed from a flexible material, such as silicone, polyurethane, thermoplastic elastomer, etc., such that the housing 420 is conformable to a contour of an anterior aspect of a patient's limb (e.g., wrist). In another embodiment, the housing 423 may be formed from a rigid or semi-rigid material, such as polycarbonate, high density polyurethane, polypropylene, etc., such that the housing is formed with a contour shape configured to conform to the anterior aspect of the patient's wrist. The housing 420 can be formed using any suitable manufacturing technique, such as injection molding, casting, 3-D printing, etc. The housing may be transparent or translucent to allow a user to view the inside of the housing 420.

The housing 420, as illustrated in the depicted embodiment of FIGS. 8A-8B, includes a side wall 422 extending distally from a top surface 431. The side wall 422 terminates at a distal sealing surface 423. The distal sealing surface 423 may include a smooth, flat surface with a width that is approximately equivalent to a thickness of the side wall 422. In other embodiments, the distal sealing surface 423 may include a flange extending radially outward from the distal sealing surface 423. The flange may be configured to increase a surface area of the sealing surface to enhance sealing of the housing 420 against the skin of a patient. In another embodiment, a compressible gasket may be coupled to the sealing surface 423 to enhance the sealability of the sealing surface 423 to the patient's skin. The gasket may be formed of any suitable low modulus, low durometer material, such as silicone, polyurethane, thermoplastic elastomer, closed cell foam, etc. The gasket can be coupled to the sealing surface 423 using any suitable manufacturing technique, such as over molding, gluing, bonding, etc. In some embodiments, a topical gel, liquid, or skin protective film may be applied to the patient's skin to achieve and maintain a leak-proof seal between the distal sealing surface 423 and the patient's skin. The side wall 422 and the top surface 431 of the housing 423 can define a suction chamber 421 along with the surface of a patient when applied to a treatment site.

The counter force member or anvil 470 is shown in the illustrated embodiment of FIGS. 8A-8B to be disposed within the suction chamber 421 and to extend distally from the top surface 431. The anvil 470 can divide the suction chamber 421 into at least two portions. The anvil 470 includes a distal end 471 which may be flush with the sealing surface 423. In other embodiments, the distal end 471 may be is recessed into the suction chamber 421. The anvil 470 may be integral with the housing 420 and formed from the same material as the housing 420. In other embodiments, the anvil 470 may be a separate component of a different material and coupled to the housing using any suitable technique, such as over molding, gluing, bonding, etc. In certain embodiments, the anvil 470 may be flexible. In other embodiments, the anvil 470 may be rigid or semi-rigid.

The anvil 470 of the embodiment illustrated in FIG. 8B extends across a longitudinal axis of the housing 420. The distal end 471 is flat and smooth. In other embodiments, the anvil 470 may be of any suitable shape such that a patient's skin, subcutaneous tissue, and artery can be drawn against the distal end 471 of the anvil 470 to close an arteriotomy. FIGS. 2A-5B depict alternative exemplary shapes of the anvil 470. Any one of the alternative exemplary anvil shapes may be used with the arteriotomy closure device 400.

The housing 420, in the illustrated embodiment of FIGS. 8A-8B, includes a port 432 disposed through the top surface 431. The port 432 is in fluid communication with both portions of the suction chamber 421. The port 432 may be disposed off-center from a center point of the top surface 431 in order to allow a user to align the housing 420 with the arteriotomy site. A valve member 480 may be coupled to the top surface 431 such that the valve member 480 is in fluid communication with the suction chamber 421 through the port 432. The valve member 480 may be configured to retain a negative gauge pressure or suction force within the suction chamber 421. In the illustrated embodiment of FIGS. 8A-8B, the valve member 480 is a check-valve 481. The check-valve 481 may be selectively opened to allow a negative gauge pressure or suction force to be formed in the suction chamber 421 and selectively closed to retain the suction force in the suction chamber 421.

As shown in FIGS. 8A-8B, the housing 420 may be coupled to a securement band 410. The securement band 410 may be configured to be disposed around a portion of a patient's limb in order to secure the housing 420 over an arteriotomy site. For example, the securement band 410 may be a wrist band configured to be disposed around a wrist of a patient such that the housing 420 can be secured over a radial or ulnar artery arteriotomy site. In other embodiments, the securement band 410 may be configured to be disposed around a patient's hand, thigh, ankle, upper arm, etc. The securement band 410 may be formed of a flexible polymeric film and include a fixation member 412 configured to selectively couple ends of the securement band 410 together when disposed around the portion of the patient's limb. For example, the fixation member 412 may be a hook and loop material, buckle, snap, button, adhesive, etc. The securement band 410 may include an aperture 413 sized to accept the housing 420 such that the housing 420 can be disposed through the aperture 413 and coupled to the securement band 410.

In use, following an arterial catheterization procedure and prior to removal of an introducer sheath, the arteriotomy closure device 400 may be positioned on a portion of a limb of a patient such that the housing 420 is disposed over an arteriotomy site. In certain embodiments, the housing 420 may be disposed over the arteriotomy site, a skin puncture site, and/or a skin tract between the arteriotomy site and the skin puncture site. The securement band 410 may be wrapped around the portion of the limb with the ends of the securement band 410 releasably coupled together. The housing 420 may be held tightly to the patient's skin such that the distal sealing surface 423 forms an airtight seal between the housing 420 and the patient's skin. In some embodiments, a liquid, gel, or skin protective film may be applied to the patient's skin prior to placement of the housing 420 to facilitate achieving the airtight seal. A syringe may be coupled to the check-valve 481. A plunger of the syringe can be displaced from a distal position to a proximal position to generate a suction force within the syringe and the suction chamber 421. The plunger may be locked in the proximal position by an optional plunger retention member. The negative gauge pressure or suction force within the suction chamber 421 may cause the patient's skin, subcutaneous tissue, and artery to be drawn, or distended, in a proximal, or puckered, position toward the housing 420 and anvil 470, such that the anvil 470 applies a counter force against the patient's skin overlying the arteriotomy and/or the tissue tract which facilitates hemostasis, or cessation of bleeding, from the arteriotomy. In certain embodiments, the suction force is formed in the suction chamber 421 prior to removal of the introducer sheath from the artery. In other embodiments, the introducer sheath is removed from the artery following forming of the suction force in the suction chamber 421.

When cessation of bleeding from the skin puncture site has been achieved, the plunger may be moved to the distal position such that the suction force in the suction chamber 421 is relieved and atmospheric pressure inside the suction chamber 421 is restored, thus removing the counter force provided by the anvil 470 against the patient's skin. At this time, the arteriotomy closure device 400 may be removed from the patient's limb.

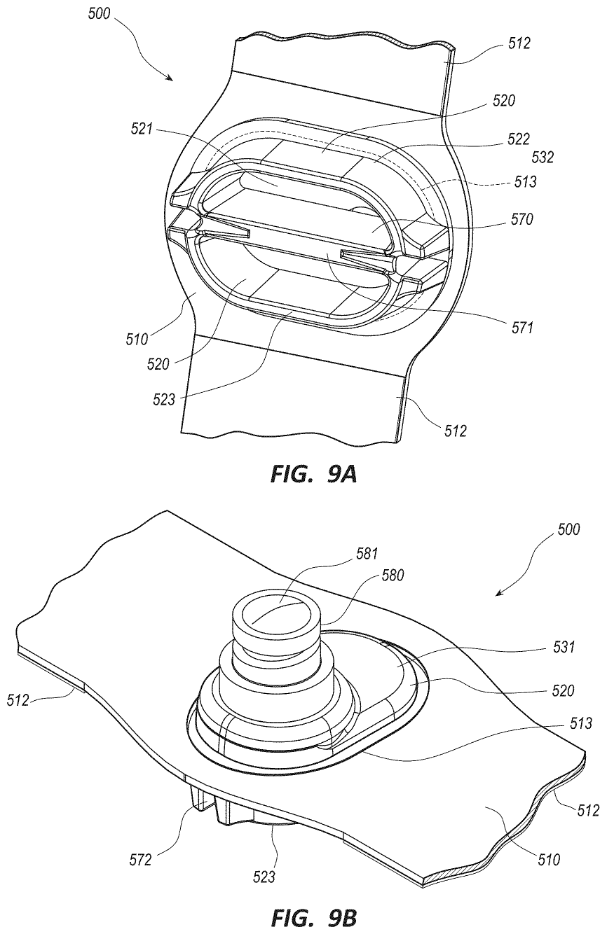

FIGS. 9A-9B illustrate an embodiment of an arteriotomy closure device 500. The arteriotomy closure device 500 includes a housing 520, a counter force member or anvil 570, a valve member 580, and a securement band 510. The housing 520 may have a generally oval or racetrack shape and may be formed from any suitable material. For example, in one embodiment, the housing 520 may be formed from a flexible material, such as silicone, polyurethane, thermoplastic elastomer, etc., such that the housing 520 is conformable to a contour of an anterior aspect of a patient's limb (e.g., wrist). In another embodiment, the housing 520 may be formed from a rigid or semi-rigid material, such as polycarbonate, high density polyurethane, polypropylene, etc., such that the housing is formed with a contour shape configured to conform to the anterior aspect of the patient's wrist. The housing 520 can be formed using any suitable manufacturing technique, such as injection molding, casting, 3-D printing, etc. The housing may be transparent or translucent to allow a user to view the inside of the housing 520.

The housing 520, as illustrated in the depicted embodiment of FIGS. 9A-9B, includes a side wall 522 extending distally from a top surface 531. The side wall 522 terminates at a distal sealing surface 523. The distal sealing surface 523 may include a smooth, flat surface with a width that is approximately equivalent to a thickness of the side wall 522. In other embodiments, the distal sealing surface 523 may include a flange extending radially outward from the distal sealing surface 523. The flange may be configured to increase a surface area of the sealing surface to enhance sealing of the housing 520 against the skin of a patient. In another embodiment, a compressible gasket may be coupled to the sealing surface 523 to enhance the sealability of the sealing surface 523 to the patient's skin. The gasket may be formed of any suitable low modulus, low durometer material, such as silicone, polyurethane, thermoplastic elastomer, closed cell foam, etc. The gasket can be coupled to the sealing surface 523 using any suitable manufacturing technique, such as over molding, gluing, bonding, etc. In some embodiments, a topical gel, liquid, or skin protective film may be applied to the patient's skin to achieve and maintain a leak-proof seal between the distal sealing surface 423 and the patient's skin. The side wall 522 and the top surface 531 of the housing 523 can define a suction chamber 521 when coupled to a treatment site of a patient.

The counter force member or anvil 570 is shown in the illustrated embodiment of FIGS. 9A-9B to be disposed within the suction chamber 521 and to extend distally from the top surface 531. The anvil 570 can divide the suction chamber 521 into at least two portions. The anvil 570 includes a distal end 571 which may be flush with the sealing surface 523. In other embodiments, the distal end 571 may be is recessed into the suction chamber 521. The anvil 570 may be integral with the housing 520 and formed from the same material as the housing 520. In other embodiments, the anvil 570 may be a separate component of a different material and coupled to the housing using any suitable technique, such as over molding, gluing, bonding, etc. In certain embodiments, the anvil 570 may be flexible. In other embodiments, the anvil 570 may be rigid or semi-rigid.

The anvil 570 of the embodiment illustrated in FIG. 9B extends across a longitudinal axis of the housing 520. The distal end 571 is flat and smooth. In other embodiments, the anvil 570 may be of any suitable shape such that a patient's skin, subcutaneous tissue, and artery can be drawn against the distal end 571 of the anvil 570 to close an arteriotomy. FIGS. 2A-5B depict alternative exemplary shapes of the anvil 570. Any one of the alternative exemplary anvil shapes may be used with the arteriotomy closure device 500.

As depicted in FIG. 9B, the anvil 570 includes a sheath channel or groove 572 disposed at both ends of the anvil 570. In another embodiment, a sheath channel 572 may be disposed at only one end of the anvil 570. The sheath channel 572 may be diametrically sized to receive an introducer sheath. The sheath channel 572 may be distally tapered from a peripheral portion of the housing 520 toward a central portion of the housing 520. The sheath channel 572 may be configured to receive the introducer sheath when the housing is disposed over the arteriotomy site such that an airtight seal may be formed around the introducer sheath. The sheath channel or groove 572 may permit the withdrawal of the elongate medical device while maintaining suction above the arteriotomy site.

The housing 520, in the illustrated embodiment of FIGS. 9A-9B, includes a port 532 disposed through the top surface 531. The port 532 is in fluid communication with both portions of the suction chamber 521. The port 532 may be disposed off-center from a center point of the top surface 531 in order to allow a user to align the housing 520 with the arteriotomy site. A valve member 580 may be coupled to the top surface 531 such that the valve member 580 is in fluid communication with the suction chamber 521 through the port 532. The valve member 580 may be configured to retain a negative gauge pressure or suction force within the suction chamber 521. In the illustrated embodiment of FIGS. 9A-9B, the valve member 580 is a check-valve 581. The check-valve 581 may be selectively opened to allow a negative gauge pressure or suction force to be formed in the suction chamber 521 and selectively closed to retain the suction force in the suction chamber 521.

As shown in FIGS. 9A-9B, the housing 520 may be coupled to a securement band 510. The securement band 510 may be configured to be disposed around a portion of a patient's limb in order to secure the housing 520 over an arteriotomy site. For example, the securement band 510 may be a wrist band configured to be disposed around a wrist of a patient such that the housing 520 can be secured over a radial or ulnar artery arteriotomy site. In other embodiments, the securement band 510 may be configured to be disposed around a patient's hand, thigh, ankle, upper arm, etc. The securement band 510 may be formed of a flexible polymeric film and include a fixation member 512 configured to selectively couple ends of the securement band 510 together when disposed around the portion of the patient's limb. For example, the fixation member 512 may be a hook and loop material, buckle, snap, button, adhesive, etc. The securement band 510 may include an aperture 513 sized to accept the housing 520 such that the housing 520 can be disposed through the aperture 513 and coupled to the securement band 510.

In use, following an arterial catheterization procedure and prior to removal of an introducer sheath, the arteriotomy closure device 500 may be positioned on a portion of a limb of a patient such that the housing 520 is disposed over an arteriotomy site. In certain embodiments, the housing 520 may be disposed over the arteriotomy site, a skin puncture site, and/or a skin tract between the arteriotomy site and the skin puncture site. The securement band 510 may be wrapped around the portion of the limb with the ends of the securement band 510 releasably coupled together. The housing 520 may be held tightly to the patient's skin such that the distal sealing surface 523 forms an airtight seal between the housing 520 and the patient's skin. In some embodiments, a liquid, gel, or skin protective film may be applied to the patient's skin prior to placement of the housing 520 to facilitate achieving the airtight seal. A syringe may be coupled to the check-valve 581. A plunger of the syringe can be displaced from a distal position to a proximal position to generate a suction force within the syringe and the suction chamber 521. The plunger may be locked in the proximal position by an optional plunger retention member. The negative gauge pressure or suction force within the suction chamber 521 may cause the patient's skin, subcutaneous tissue, and artery to be drawn, or distended, in a proximal, or puckered, position toward the housing 520 and anvil 570, such that the anvil 570 applies a counter force against the patient's skin overlying the arteriotomy and/or the tissue tract which facilitates hemostasis, or cessation of bleeding, from the arteriotomy. In certain embodiments, the suction force is formed in the suction chamber 521 prior to removal of the introducer sheath from the artery. In other embodiments, the introducer sheath is removed from the artery following forming of the suction force in the suction chamber 521.

When cessation of bleeding from the skin puncture site has been achieved, the plunger may be moved to the distal position such that the suction force in the suction chamber 521 is relieved and atmospheric pressure inside the suction chamber 521 is restored, thus removing the counter force provided by the anvil 570 against the patient's skin. At this time, the arteriotomy closure device 500 may be removed from the patient's limb.

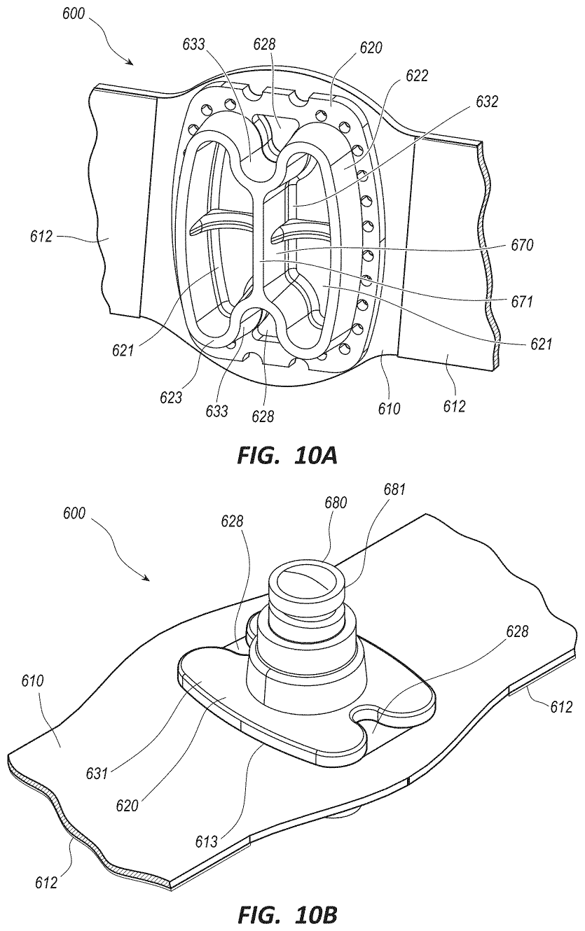

FIGS. 10A-10B illustrate an embodiment of an arteriotomy closure device 600. The arteriotomy closure device 600 includes a housing 620, a counter force member or anvil 670, a valve member 680, and a securement band 610. The housing 620 may have a dual oval or butterfly shape and may be formed from any suitable material. For example, in one embodiment, the housing 620 may be formed from a flexible material, such as silicone, polyurethane, thermoplastic elastomer, etc., such that the housing 620 is conformable to a contour of an anterior aspect of a patient's limb (e.g., wrist). In another embodiment, the housing 620 may be formed from a rigid or semi-rigid material, such as polycarbonate, high density polyurethane, polypropylene, etc., such that the housing is formed with a contour shape configured to conform to the anterior aspect of the patient's wrist. The housing 620 can be formed using any suitable manufacturing technique, such as injection molding, casting, 3-D printing, etc. The housing may be transparent or translucent to allow a user to view the inside of the housing 620.

The housing 620, as illustrated in the depicted embodiment of FIGS. 10A-10B, includes a side wall 622 extending distally from a top surface 631. The side wall 622 terminates at a distal sealing surface 623. The distal sealing surface 623 may include a smooth, flat surface with a width that is approximately equivalent to a thickness of the side wall 622. In other embodiments, the distal sealing surface 623 may include a flange extending radially outward from the distal sealing surface 623. The flange may be configured to increase a surface area of the sealing surface to enhance sealing of the housing 620 against the skin of a patient. In another embodiment, a compressible gasket may be coupled to the sealing surface 623 to enhance the sealability of the sealing surface 623 to the patient's skin. The gasket may be formed of any suitable low modulus, low durometer material, such as silicone, polyurethane, thermoplastic elastomer, closed cell foam, etc. The gasket can be coupled to the sealing surface 623 using any suitable manufacturing technique, such as over molding, gluing, bonding, etc. In some embodiments, a topical gel, liquid, or skin protective film may be applied to the patient's skin to achieve and maintain a leak-proof seal between the distal sealing surface 623 and the patient's skin. The side wall 622 and the top surface 631 of the housing 620 can define a suction chamber 621 when coupled to a treatment site of a patient.

The housing 620 may include a window 628 disposed at either end of the housing 620. In other embodiments, the housing 620 may include a single window 628 disposed at one end of the housing 620. The window 628 defines an aperture through the top surface 631 of the housing 620. The side wall 622 may be recessed radially inward to form a viewing channel 633 beneath the window 628. The window 628 may be configured to allow a user to position the housing 620 proximal to a skin puncture site by viewing the skin puncture site through the window 628 and the viewing channel 633. In some embodiments the skin puncture site is disposed below the window 628 but outside of the suction chamber 621. In other embodiments, the skin puncture site is disposed inside the suction chamber 621.

The counter force member or anvil 670 is shown in the illustrated embodiment of FIG. 10B to be disposed within the suction chamber 621 and to extend distally from the top surface 631. The anvil 670 can divide the suction chamber 621 into at least two portions. The anvil 670 includes a distal end 671 which may be flush with the sealing surface 623. In other embodiments, the distal end 671 may be recessed into the suction chamber 621. The anvil 670 may be integral with the housing 620 and formed from the same material as the housing 620. In other embodiments, the anvil 670 may be a separate component of a different material and coupled to the housing using any suitable technique, such as over molding, gluing, bonding, etc. In certain embodiments, the anvil 670 may be flexible. In other embodiments, the anvil 670 may be rigid or semi-rigid.

The anvil 670 of the embodiment illustrated in FIG. 10B extends across a longitudinal axis of the housing 620. The distal end 671 is flat and smooth. In other embodiments, the anvil 670 may be of any suitable shape such that a patient's skin, subcutaneous tissue, and artery can be drawn against the distal end 671 of the anvil 670 to close an arteriotomy. FIGS. 2A-5B depict alternative exemplary shapes of the anvil 670. Any one of the alternative exemplary anvil shapes may be used with the arteriotomy closure device 600.

The housing 620, in the illustrated embodiment of FIGS. 10A-10B, includes a port 632 disposed through the top surface 631. The port 632 is in fluid communication with both portions of the suction chamber 621. A valve member 680 may be coupled to the top surface 631 such that the valve member 680 is in fluid communication with the suction chamber 621 through the port 632. The valve member 680 may be configured to retain a negative gauge pressure or suction force within the suction chamber 621. In the illustrated embodiment of FIGS. 10A-10B, the valve member 680 is a check-valve 681. The check-valve 681 may be selectively opened to allow a negative gauge pressure or suction force to be formed in the suction chamber 621 and selectively closed to retain the suction force in the suction chamber 621.

As shown in FIGS. 10A-10B, the housing 620 may be coupled to a securement band 610. The securement band 610 may be configured to be disposed around a portion of a patient's limb in order to secure the housing 620 over an arteriotomy site. For example, the securement band 610 may be a wrist band configured to be disposed around a wrist of a patient such that the housing 620 can be secured over a radial or ulnar artery arteriotomy site. In other embodiments, the securement band 610 may be configured to be disposed around a patient's hand, thigh, ankle, upper arm, etc. The securement band 610 may be formed of a flexible polymeric film and include a fixation member 612 configured to selectively couple ends of the securement band 610 together when disposed around the portion of the patient's limb. For example, the fixation member 612 may be a hook and loop material, buckle, snap, button, adhesive, etc. The securement band 610 may include an aperture 613 sized to accept the housing 620 such that the housing 620 can be disposed through the aperture 613 and coupled to the securement band 610.

In use, following an arterial catheterization procedure and prior to removal of an introducer sheath, the arteriotomy closure device 600 may be positioned on a portion of a limb of a patient such that the housing 620 is disposed over an arteriotomy site. In certain embodiments, the housing 620 may be disposed over the arteriotomy site, a skin puncture site, and/or a skin tract between the arteriotomy site and the skin puncture site. The window 628 may be utilized to position the housing 620 proximal to the skin puncture site. The securement band 610 may be wrapped around the portion of the limb with the ends of the securement band 610 releasably coupled together. The housing 620 may be held tightly to the patient's skin such that the distal sealing surface 623 forms an airtight seal between the housing 620 and the patient's skin. In some embodiments, a liquid, gel, or skin protective film may be applied to the patient's skin prior to placement of the housing 620 to facilitate achieving the airtight seal. A syringe may be coupled to the check-valve 681. A plunger of the syringe can be displaced from a distal position to a proximal position to generate a negative gauge pressure or suction force within the syringe and the suction chamber 621. The plunger may be locked in the proximal position by an optional plunger retention member. The suction force within the suction chamber 621 may cause the patient's skin, subcutaneous tissue, and artery to be drawn, or distended, in a proximal, or puckered, position toward the housing 620 and anvil 670, such that the anvil 670 applies a counter force against the patient's skin overlying the arteriotomy and/or the tissue tract which facilitates hemostasis, or cessation of bleeding, from the arteriotomy. In certain embodiments, the suction force is formed in the suction chamber 621 prior to removal of the introducer sheath from the artery. In other embodiments, the introducer sheath is removed from the artery following forming of the suction force in the suction chamber 621.

When cessation of bleeding from the skin puncture site has been achieved, the plunger may be moved to the distal position such that the suction force in the suction chamber 621 is relieved and atmospheric pressure inside the suction chamber 621 is restored, thus removing the counter force provided by the anvil 670 against the patient's skin. At this time, the arteriotomy closure device 600 may be removed from the patient's limb.

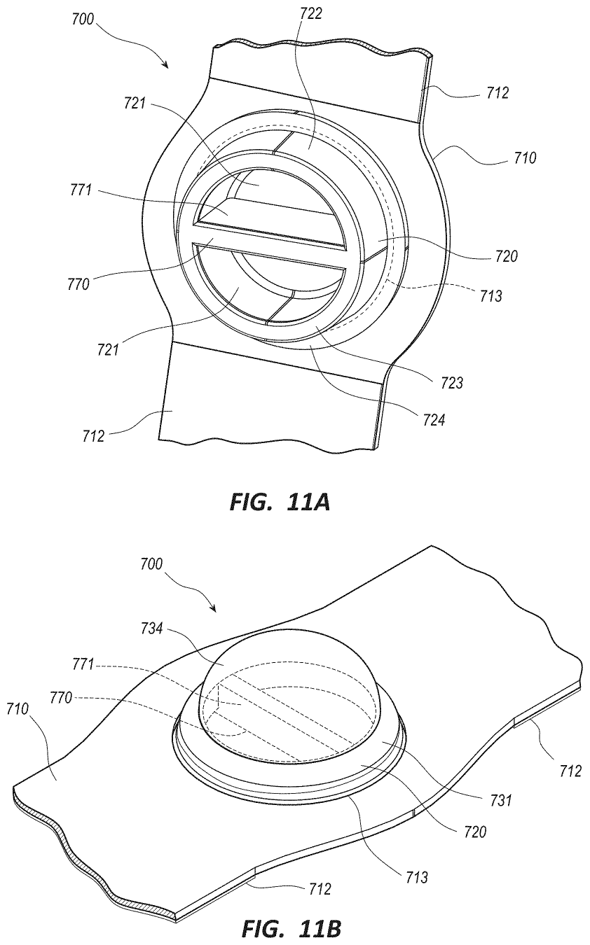

FIGS. 11A-11B illustrate an embodiment of an arteriotomy closure device 700. The arteriotomy closure device 700 includes a housing 720, a counter force member or anvil 770, and a securement band 710. The housing 720 may have a generally circular shape and may be formed from any suitable material. For example, in one embodiment, the housing 720 may be formed from a flexible material, such as silicone, polyurethane, thermoplastic elastomer, etc., such that the housing 720 is conformable to a contour of an anterior aspect of a patient's limb (e.g., wrist). In another embodiment, the housing 720 may be formed from a rigid or semi-rigid material, such as polycarbonate, high density polyurethane, polypropylene, etc., such that the housing is formed with a contour shape configured to conform to the anterior aspect of the patient's wrist. The housing 720 can be formed using any suitable manufacturing technique, such as injection molding, casting, 3-D printing, etc. The housing may be transparent or translucent to allow a user to view the inside of the housing 720.

The housing 720, as illustrated in the depicted embodiment of FIGS. 7A-7B, includes a side wall 722 extending distally from a top surface 731. The side wall 722 terminates at a distal sealing surface 723. In some embodiments, a topical gel, liquid, or skin protective film may be applied to the patient's skin to achieve and maintain a leak-proof seal between the distal sealing surface 723 and the patient's skin. The side wall 722 and the top surface 731 of the housing 720 can define a suction chamber 721. The housing 720 may include a flange 724 extending radially outward from the housing 720. The flange 724 may be configured to assist in securing the housing to a securement band 710.

The counter force member or anvil 770 is shown in the illustrated embodiment of FIGS. 11A-11B to be disposed within the suction chamber 721 and to extend distally from the top surface 731. The anvil 770 can divide the suction chamber 321 into at least two portions. The anvil 770 includes a distal end 771 which is flush or co-planar with the sealing surface 723. In other embodiments, the distal end 771 of the anvil 770 may be recessed into the suction chamber 721 such that the sealing surface 723 extends distally beyond the distal end 771 of the anvil 770. The anvil 770 may be integral with the housing 720 and formed from the same material as the housing 720. In other embodiments, the anvil 770 may be a separate component of a different material and coupled to the housing using any suitable technique, such as over molding, gluing, bonding, etc. In certain embodiments, the anvil 770 may be flexible. In other embodiments, the anvil 770 may be rigid or semi-rigid.

The anvil 770 of the embodiment illustrated in FIG. 11B extends across a diameter of the housing 720. The distal end 771 is flat and smooth. In other embodiments, the anvil 770 may be of any suitable shape such that a patient's skin, subcutaneous tissue, and artery can be drawn against the distal end 771 of the anvil 770 to close an arteriotomy. FIGS. 2A-5B depict exemplary shapes of the anvil 770. Any one of the exemplary anvil shapes may be used with the arteriotomy closure device 700.

As shown in FIGS. 11A-11B, the housing 720 may be coupled to a securement band 710. The securement band 710 may be configured to be disposed around a portion of a patient's limb in order to secure the housing 720 over an arteriotomy site. For example, the securement band 710 may be a wrist band configured to be disposed around a wrist of a patient such that the housing 720 can be secured over a radial or ulnar artery arteriotomy site. In other embodiments, the securement band 710 may be configured to be disposed around a patient's hand, thigh, ankle, upper arm, etc. The securement band 710 may be formed of a flexible polymeric film and include a fixation member 712 configured to selectively couple ends of the securement band 710 together when disposed around the portion of the patient's limb. For example, the fixation member 712 may be a hook and loop material, buckle, snap, button, adhesive, etc. The securement band 710 may include an aperture 713 sized to accept the housing 720 such that the housing 720 can be disposed through the aperture 713 and coupled to the securement band 710.

In use, following an arterial catheterization procedure and prior to removal of an introducer sheath, the arteriotomy closure device 700 may be positioned on a portion of a limb of a patient such that the housing 720 is disposed over an arteriotomy site. In certain embodiments, the housing 720 may be disposed over the arteriotomy site, a skin puncture site, and/or a skin tract between the arteriotomy site and the skin puncture site. The securement band 710 may be wrapped around the portion of the limb with the ends of the securement band 710 releasably fixed together. The housing 720 may be held tightly to the patient's skin such that the distal sealing surface 723 forms an airtight seal between the housing 720 and the patient's skin. In some embodiments, a liquid, gel, or skin protective film may be applied to the patient's skin prior to placement of the housing 720 to facilitate achieving the airtight seal. A negative gauge pressure or suction force may be formed in the suction chamber 721 when a user depresses the housing 720 toward the patient's skin. When depressed, the housing 720 may collapse forcing air out of the suction chamber 721. When released, the housing 720 may return to a non-collapsed configuration where the sealing surface 723 has formed an airtight seal to the skin surface and a negative gauge pressure or suction force is contained within the suction chamber 721. The suction force within the suction chamber 721 may cause the patient's skin, subcutaneous tissue, and artery to be drawn, or distended, in a proximal, or puckered, position toward the housing 720 and anvil 770, such that the anvil 770 applies a counter force against the patient's skin overlying the arteriotomy and/or the tissue tract which facilitates hemostasis, or cessation of bleeding, from the arteriotomy. In certain embodiments, the suction force is formed in the suction chamber 721 prior to removal of the introducer sheath from the artery. In other embodiments, the introducer sheath is removed from the artery following forming of the suction force in the suction chamber 721.

When cessation of bleeding from the skin puncture site has been achieved, the housing 720 may be partly lifted from the patient's skin such that the airtight seal is broken and the suction force in the suction chamber 721 is relieved and atmospheric pressure inside the suction chamber 721 is restored, thus removing the counter force provided by the anvil 770 against the patient's skin. At this time, the arteriotomy closure device 700 may be removed from the patient's limb.

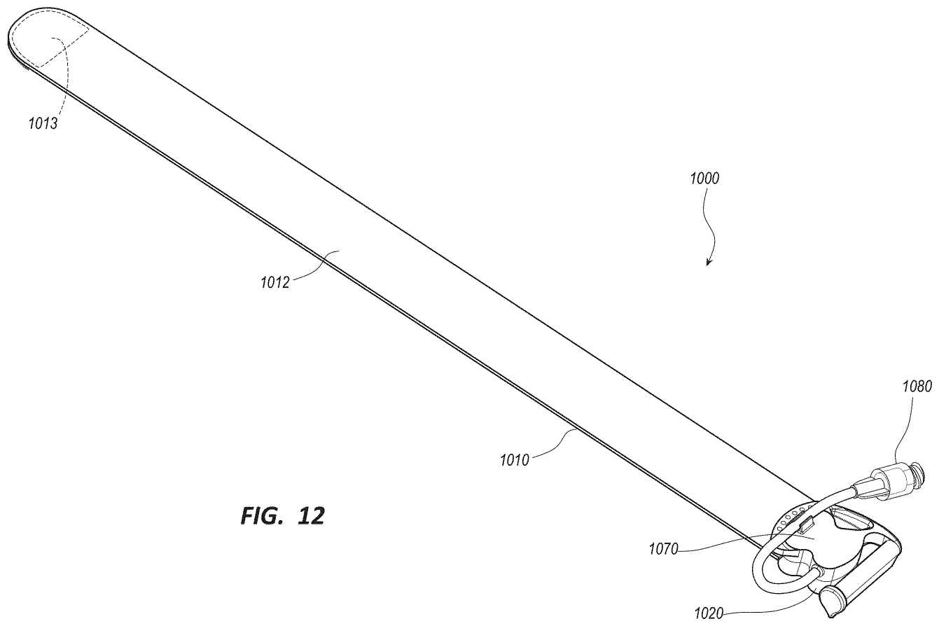

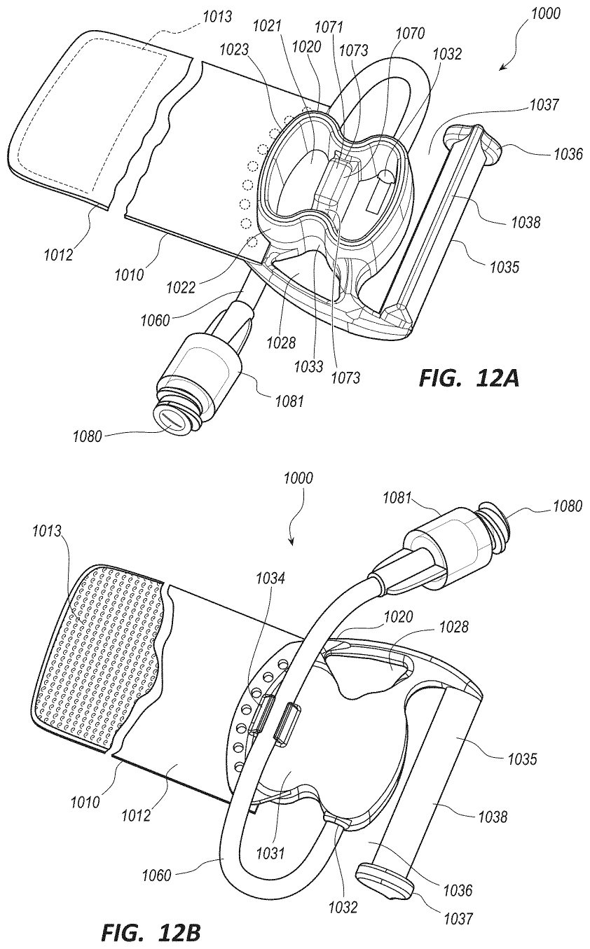

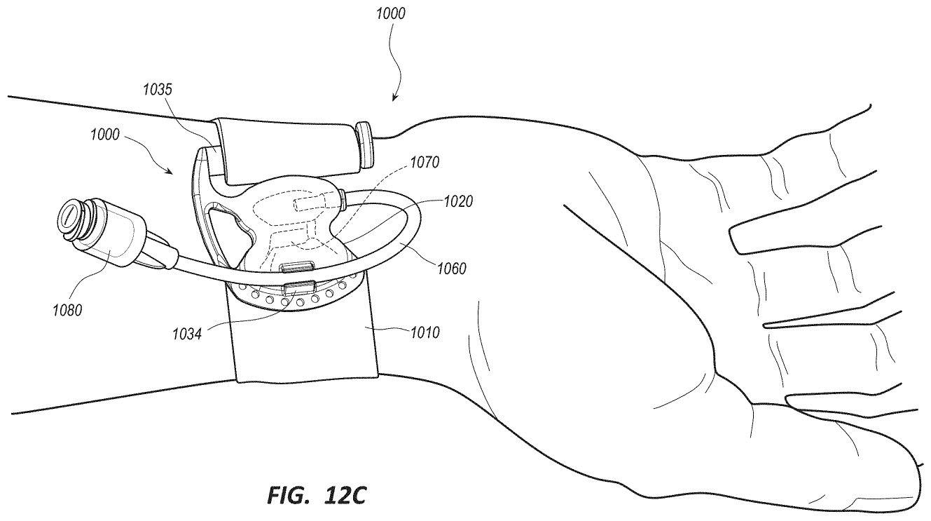

FIGS. 12-12C illustrate an embodiment of an arteriotomy closure device 1000. The arteriotomy closure device 1000 includes a housing 1020, a counter force member or anvil 1070, a valve member 1080, and a securement band 1010. The housing 1020 may have a dual oval or butterfly shape. For example, in one embodiment, the housing 1020 may be formed from a flexible material, such as silicone, polyurethane, thermoplastic elastomer, etc., such that the housing 1020 is conformable to a contour of an anterior aspect of a patient's limb (e.g., wrist). In another embodiment, the housing 1020 may be formed from a rigid or semi-rigid material, such as polycarbonate, high density polyurethane, polypropylene, etc., such that the housing is formed with a contour shape configured to conform to the anterior aspect of the patient's wrist. The housing 1020 can be formed using any suitable manufacturing technique, such as injection molding, casting, 3-D printing, etc. The housing may be transparent or translucent to allow a user to view the inside of the housing 1020.

The housing 1020, as illustrated in the depicted embodiment of FIGS. 12A-12B, includes a side wall 1022 extending distally from a top surface 1031. The side wall 1022 terminates at a distal sealing surface 1023. The distal sealing surface 1023 may include a smooth, flat surface with a width that is approximately equivalent to a thickness of the side wall 1022. In other embodiments, the distal sealing surface 1023 may include a flange extending radially outward from the distal sealing surface 1023 or side wall 1022. The flange may be configured to increase a surface area of the sealing surface to enhance sealing of the housing 1020 against the skin of a patient. In another embodiment, a compressible gasket may be coupled to the sealing surface 1023 to enhance the sealability of the sealing surface 1023 to the patient's skin. The gasket may be formed of any suitable low modulus, low durometer material, such as silicone, polyurethane, thermoplastic elastomer, closed cell foam, etc. The gasket can be coupled to the sealing surface 1023 using any suitable manufacturing technique, such as over molding, gluing, bonding, etc. In some embodiments, a topical gel, liquid, or skin protective film may be applied to the patient's skin to achieve and maintain a leak-proof seal between the distal sealing surface 1023 and the patient's skin. The side wall 1022 and the top surface 1031 of the housing 1020 can define a suction chamber 1021 when coupled to a treatment site of a patient.

The housing 1020 may include a window 1028 disposed at either end of the housing 1020. In other embodiments, the housing 1020 may include a single window 1028 disposed at one end of the housing 1020. The window 1028 defines an aperture through the top surface 1031 of the housing 1020. The side wall 1022 may be recessed radially inward to form a viewing channel 1033 beneath the window 1028. The window 1028 may be configured to allow a user to position the housing 1020 proximal to a skin puncture site by viewing the skin puncture site through the window 1028 and the viewing channel 1033. In some embodiments the skin puncture site is disposed below the window 1028 but outside of the suction chamber 1021. In other embodiments, the skin puncture site is disposed inside the suction chamber 1021.

The counter force member or anvil 1070 is shown in the illustrated embodiment of FIG. 12A to be disposed within the suction chamber 1021 and to extend distally from the top surface 1031. The anvil 1070 can divide the suction chamber 1021 into at least two portions. A flow channel 1073 may be disposed on either end of the anvil 1070 such that a suction force can be formed in the portions of the suction chamber 1021. The anvil 1070 includes a distal end 1071 which may be flush with the sealing surface 1023. In other embodiments, the distal end 1071 may be recessed into the suction chamber 1021. The anvil 1070 may be integral with the housing 1020 and formed from the same material as the housing 1020. In other embodiments, the anvil 1070 may be a separate component of a different material and coupled to the housing using any suitable technique, such as over molding, gluing, bonding, etc. In certain embodiments, the anvil 1070 may be flexible. In other embodiments, the anvil 1070 may be rigid or semi-rigid.

The anvil 1070 of the embodiment illustrated in FIG. 12A extends across a longitudinal axis of the housing 1020. The distal end 1071 of the anvil 1070 is flat and smooth. In other embodiments, the anvil 1070 may be of any suitable shape such that a patient's skin, subcutaneous tissue, and artery can be drawn against the distal end 1071 to close an arteriotomy. FIGS. 2A-5B depict alternative exemplary shapes of the anvil 1070. Any one of the alternative exemplary anvil shapes may be used with the arteriotomy closure device 1000.

The housing 1020, in the illustrated embodiment of FIGS. 12A-12B, includes a port 1032 disposed through the side wall 1022. In other embodiments, the port 1032 may be disposed through the top surface 1031. The port 1032 is in fluid communication with both portions of the suction chamber 1021. An extension tube 1060 may be coupled to the port 1032 at one end. A C-shaped tubing holder 1034 may be optionally disposed on the top surface 1031 to releasably secure the tubing 1032 to the housing 1020. A valve member 1080 may be coupled to the extension tube 1060 at an opposite end such that the valve member 1080 is in fluid communication with the suction chamber 1021 through the extension tube 1060. The position of the valve member 1080 at the end of the extension tube 1060 allows the valve member to be accessed by a syringe or other medical device without breaking the seal formed between the housing 1020 and the patient's skin which may result in a loss of the suction force. The valve member 1080 may be configured to retain a negative gauge pressure or suction force within the suction chamber 1021. In the illustrated embodiment of FIGS. 12A-12B, the valve member 1080 is a check-valve 1081. The check-valve 1081 may be selectively opened to allow a negative gauge pressure or suction force to be formed in the suction chamber 1021 and selectively closed to retain the suction force in the suction chamber 1021.

The housing 1020 includes a horizontally oriented hook 1035 disposed at an end of the housing 1020 opposite the securement band 1010. In one embodiment, the hook 1035 may be integrally formed with the housing 1020 and formed from the same materials of the housing 1020. In another embodiment, the hook 1035 may be formed as a separate component from a different material and coupled to the housing 1020 using any suitable technique, such as over molding, gluing, bonding, etc. For example, the hook 1035 may be formed from a rigid material while the housing 1020 may be formed from a compliant material. The hook 1035 includes a bar 1039 extending in a horizontal orientation parallel to the longitudinal axis of the housing 1020 and from one side of the housing 1020. A free end 1036 of the bar 1039 may include a flange having a larger width dimension than the bar 1039. A length of the bar 1039 may be sized to accommodate a width of the securement band 1010. In the illustrated embodiment of FIGS. 12A-12B, the bar 1039 includes a radiused surface on an upper side and a spine 1038 on a lower side. The spine 1038 may be configured to secure the securement band 1010 without slippage. In other embodiments, the bar 1039 may include a cylindrical, square, triangular, oval shape, or other geometries are likewise within the scope of this disclosure. Furthermore, the bar 1039 could be part of a buckle. The bar 1039 can be disposed a distance from the wall 1022 of the housing 1020 such that a space 1037 extends from the free end 1036 to an opposite end of the bar 1039. The space 1037 is sized such that the securement band 1010 can be freely passed through the space 1037. The hook 1035 may permit a clinician to more securely dispose the housing 1020 on the patient's limb which may result in maintenance of a negative gauge pressure or suction force within the suction chamber 1021.

As shown in FIGS. 12-12C, the housing 1020 may be coupled to a securement band 1010. The securement band 1010 may be configured to be disposed around a portion of a patient's limb in order to secure the housing 1020 over an arteriotomy site. For example, the securement band 1010 may be a wrist band configured to be disposed around a wrist of a patient such that the housing 1020 can be secured over a radial or ulnar artery arteriotomy site. In other embodiments, the securement band 1010 may be configured to be disposed around a patient's hand, thigh, ankle, upper arm, etc. The securement band 1010 may be formed of a flexible polymeric film and include a fixation member 1012 and a fastener 1013 disposed at a free end on the securement band 1010. The fixation member 1012 and the fastener 1013 are configured to selectively couple the free end of the securement band 1010 with a portion of the securement band 1010 when disposed around the portion of the patient's limb and around the hook 1035. For example, the fixation member 1012 and the fastener 1013 may be a hook and loop material, snap, button, adhesive, etc. An end of the securement band 1010 opposite the free end may be fixedly coupled to the housing 1020 using any suitable technique, such as gluing, welding, bonding, etc.

In use, as depicted in FIG. 12C, following an arterial catheterization procedure and prior to removal of an introducer sheath, the arteriotomy closure device 1000 may be positioned on a portion of a limb of a patient such that the housing 1020 is disposed over an arteriotomy site. In certain embodiments, the housing 1020 may be disposed over the arteriotomy site, a skin puncture site, and/or a skin tract between the arteriotomy site and the skin puncture site. The window 1028 may be utilized to position the housing 1020 proximal to the skin puncture site. The securement band 1010 may be wrapped around the portion of the limb in a first direction. The free end of the securement band 1010 may be passed around the bar 1039 of the hook 1035 from a lower side to an upper side and then wrapped partially around the portion of the limb in an opposite direction from the first direction thereby cinching the securement band 1010 and securing the distal sealing surface 1023 to the patient's skin. In another embodiment, a loop or buckle may be formed with the securement band 1010 and the loop passed over the free end of the hook 1035 The securement band 1010 may be cinched securely and the fastener 1013 coupled to the fixation member 1012 such that the housing 1020 may be held tightly to the patient's skin such that the distal sealing surface 1023 forms an airtight seal between the housing 1020 and the patient's skin. In some embodiments, a liquid, gel, or skin protective film may be applied to the patient's skin prior to placement of the housing 1020 to facilitate achieving the airtight seal.

A syringe may be coupled to the check-valve 1081. A plunger of the syringe can be displaced from a distal position to a proximal position to generate a negative gauge pressure or suction force within the syringe and the suction chamber 1021. The plunger may be locked in the proximal position by an optional plunger retention member. The suction force within the suction chamber 1021 may cause the patient's skin, subcutaneous tissue, and artery to be drawn, or distended, in a proximal, or puckered, position toward the housing 1020 and anvil 1070, such that the anvil 1070 applies a counter force against the patient's skin overlying the arteriotomy and/or the tissue tract which facilitates hemostasis, or cessation of bleeding, from the arteriotomy. In certain embodiments, the suction force is formed in the suction chamber 1021 prior to removal of the introducer sheath from the artery. In other embodiments, the introducer sheath is removed from the artery following forming of the suction force in the suction chamber 1021.

When cessation of bleeding from the skin puncture site has been achieved, the plunger may be moved to the distal position such that the suction force in the suction chamber 1021 is relieved and atmospheric pressure inside the suction chamber 1021 is restored, thus removing the counter force provided by the anvil 1070 against the patient's skin. At this time, the arteriotomy closure device 1000 may be removed from the patient's limb.

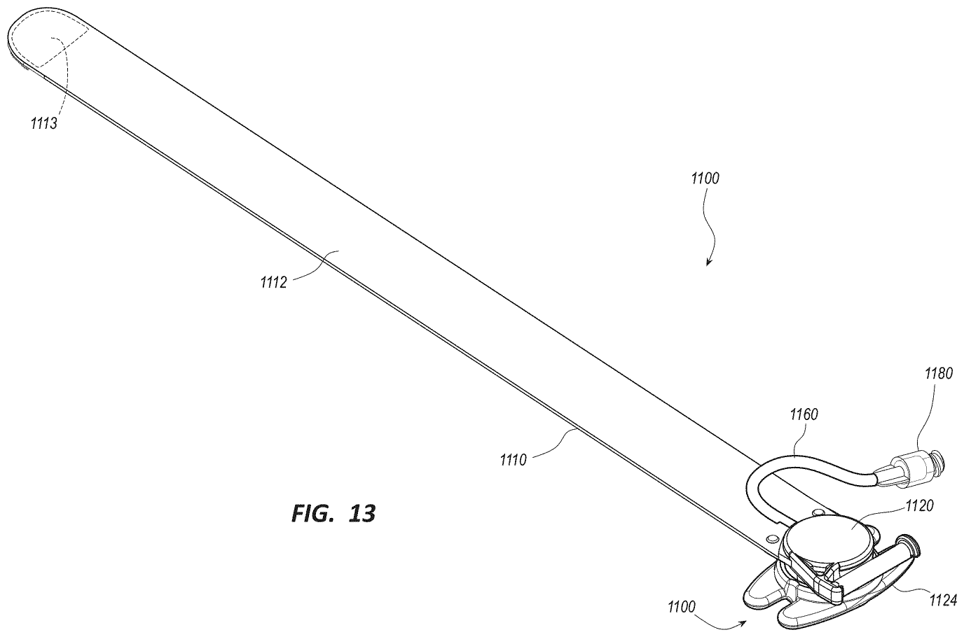

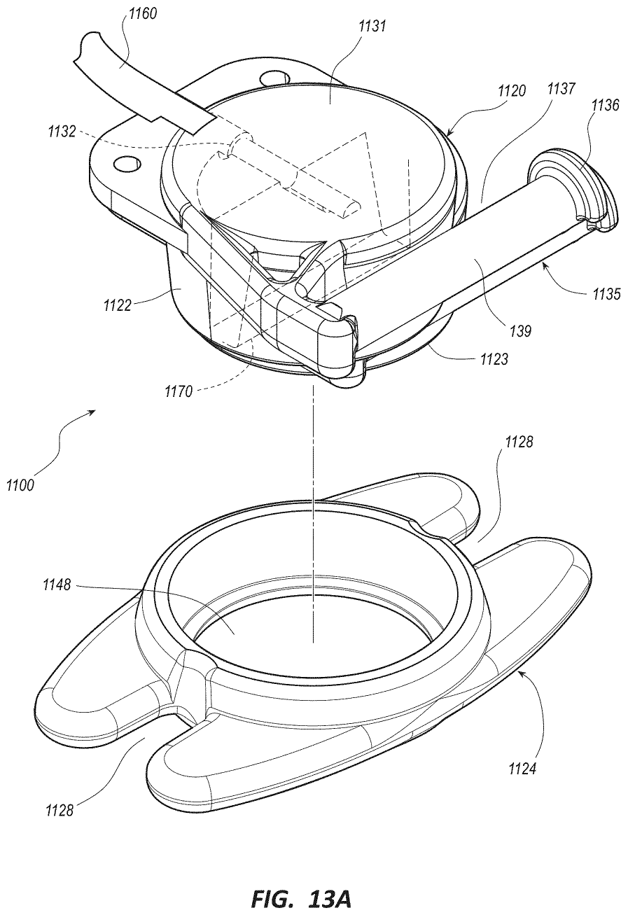

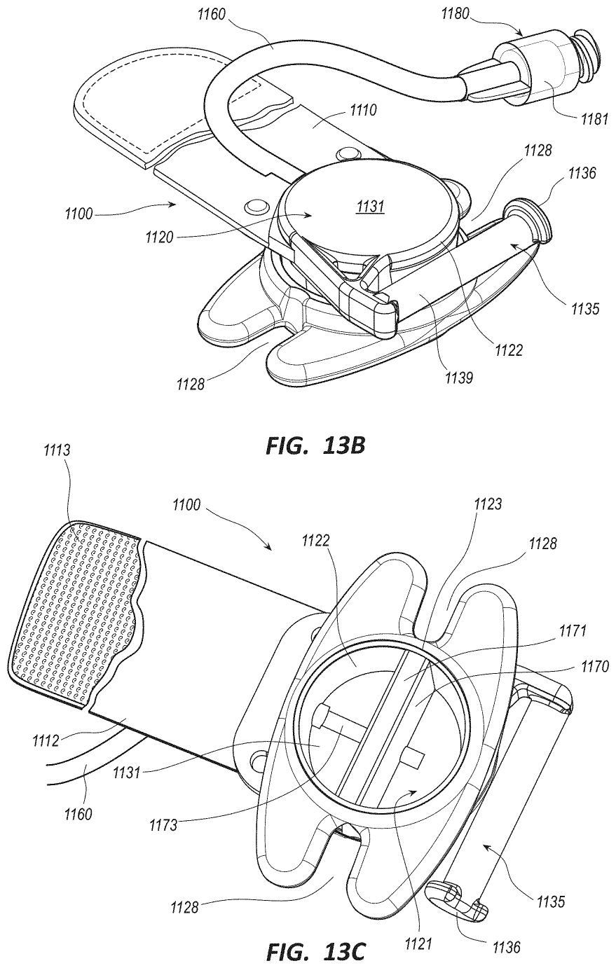

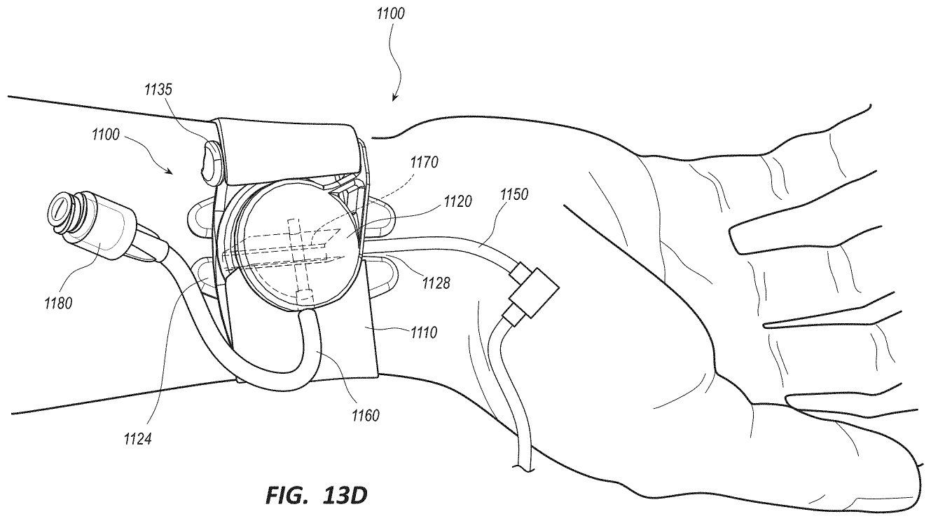

FIGS. 13-13D illustrate an embodiment of an arteriotomy closure device 1100. The arteriotomy closure device 1100 includes a housing 1120, a flange 1124, a counter force member or anvil 1170, a valve member 1180, and a securement band 1110. The housing 1120 may be elliptical or have a circular shape. For example, in one embodiment, the housing 1120 may be formed from a flexible material, such as silicone, polyurethane, thermoplastic elastomer, etc., such that the housing 1120 is conformable to a contour of an anterior aspect of a patient's limb (e.g., wrist). In another embodiment, the housing 1120 may be formed from a rigid or semi-rigid material, such as polycarbonate, high density polyurethane, polypropylene, etc., such that the housing is formed with a contour shape configured to conform to the anterior aspect of the patient's wrist. The housing 1120 can be formed using any suitable manufacturing technique, such as injection molding, casting, 3-D printing, etc. The housing may be transparent or translucent to allow a user to view the inside of the housing 1120.

The housing 1120, as illustrated in the depicted embodiment of FIGS. 13A-13C, includes a side wall 1022 extending distally from a top surface 1131. The side wall 1122 terminates at a distal sealing surface 1123. The distal sealing surface 1123 may include a smooth, flat surface with a width that is approximately equivalent to a thickness of the side wall 1022. In the present embodiment, the housing 1120 is sized to engage the flange 1124, which may include an orifice 1148 shaped to receive and retain the housing 1120. Once the housing 1120 is seated in the flange orifice 1148 it can be affixed using adhesives, optionally UV curable. Alternative seating options could include an interference fit, threaded engagement and the like.

The flange 1124 may extend radially outward from the distal sealing surface 1123 or side wall 1122. The present configuration has the flange 1124 extending from the side wall 1122 just above the distal sealing surface 1123. Stated another way, the flange 1124 may occupy a plane that is parallel to, but offset from the plane formed from the distal sealing surface 1123. The flange 1124 may be configured to increase a surface area of the sealing surface to enhance sealing of the housing 1120 against the skin of a patient. For example, when the arteriotomy closure device 1100 is secured to a limb of a patient by cinching the securement band 1110, a compressive force is imposed on the closure device 1100 such that both the distal sealing surface 1123 and the offset flange 1124 contact the patient's skin. Skin will conform around the distal sealing surface 1123 and abut the flange 1124. The flange 1124 can function to limit the patient's skin and subcutaneous tissue from compressing further underneath the distal sealing surface 1123 and inhibit or prevent manual arterial occlusion. Furthermore, it has been observed that the offset flange 1124 does not disrupt suction as much as it would if it were flush with the distal sealing surface 1123. However, under certain conditions, both flush and offset flanges may provide for effective hemostasis in practice.

The flange 124 may include a window 1128 to facilitate proper placement of the arteriotomy closure device 1100 over the arteriotomy both visually (unobstructed view) and physically, by locating the implanted elongate medical device (e.g., introducer sheath 1150) between the wings of the flange 1124 that form the window 1128. Consequently, when placed in the window 1128, the introducer sheath 1150 does not disrupt suction or negative gauge pressure. In some embodiments the skin puncture site is disposed below the window 1128 but outside of the suction chamber 1121. In other embodiments, the skin puncture site is disposed inside the suction chamber 1121.

Furthermore, the anvil 1170 may be axially aligned with the window 1128 so that placing the introducer sheath 1150 in the window 1128 positions the anvil 1170 over the arteriotomy. The flange 1124 can also provide comfort to the patient by reducing markings and skin damage that could be caused by the suction applied to the patient's skin. This is accomplished by distributing the force against the skin across a larger area when applying negative gauge pressure and/or when cinching the securement band 1110 and securing the housing 1120 to the skin.

The flange 1124 may be constructed of the same material as the housing 1120 or a material with a similar durometer hardness as the housing 1120. In other embodiments, the flange 1124 may be more rigid than the housing 1120. In yet other embodiments, the flange 1124 may be less rigid than the housing 1120.

In another embodiment, a compressible gasket may be coupled to the sealing surface 1123 to enhance the sealability of the sealing surface 1123 to the patient's skin. The gasket may be formed of any suitable low modulus, low durometer material, such as silicone, polyurethane, thermoplastic elastomer, closed cell foam, etc. The gasket can be coupled to the sealing surface 1123 using any suitable manufacturing technique, such as over molding, gluing, bonding, etc. In some embodiments, a topical gel, liquid, or skin protective film may be applied to the patient's skin to achieve and maintain a leak-proof seal between the distal sealing surface 1123 and the patient's skin. The side wall 1122 and the top surface 1131 of the housing 1120 can define a suction chamber 1121 when coupled to a treatment site of a patient.

The counter force member or anvil 1170 is shown in the illustrated embodiment of FIG. 13C to be disposed within the suction chamber 1121 and to extend distally from the top surface 1131. The anvil 1170 can divide the suction chamber 1121 into at least two portions. A flow channel 1173 may be disposed through the anvil 1170 such that a suction force or negative gauge pressure can be formed in both portions of the suction chamber 1121. The anvil 1170 includes a distal end 1171 which may be recessed into the suction chamber 1121 compared to the distal sealing surface 1123. In other embodiments, the distal end 1171 of the anvil 1170 may be flush with the distal sealing surface 1123. In has been observed in certain applications, that long-term suction can be maintained more easily with a recessed anvil 1170 than a flush anvil 1170. The anvil 1170 may be integral with the housing 1120 and formed from the same material as the housing 1120. In other embodiments, the anvil 1170 may be a separate component of a different material and coupled to the housing using any suitable technique, such as over molding, gluing, bonding, etc. In certain embodiments, the anvil 1170 may be flexible. In other embodiments, the anvil 1170 may be rigid or semi-rigid.

The anvil 1170 of the embodiment illustrated in FIG. 13C extends across a longitudinal axis of the housing 1120. In one embodiment, the anvil 1170 length is similar to that of the housing 1120, and can be between 0.5 inches and 1.0 inches. In other embodiments the anvil 1170 length (and the housing 1120 diameter) is between 0.5 inches and 0.75 inches. The distal end 1171 of the anvil 1170 may be flat and smooth. In other embodiments, the anvil 1170 may be of any suitable shape such that a patient's skin, subcutaneous tissue, and artery can be drawn against the distal end 1171 to close an arteriotomy. FIGS. 2A-5B depict alternative exemplary shapes of the anvil 1170. Any one of the alternative exemplary anvil shapes may be used with the arteriotomy closure device 1100.

The housing 1120, in the illustrated embodiment of FIGS. 13-13D, includes a port 1132 disposed through the side wall 1122 to enhance visibility compared to valves or ports located on the top surface 1131 of the housing 1120. In other embodiments, the port 1132 may be disposed through the top surface 1131. The port 1132 is in fluid communication with both portions of the suction chamber 1121. An extension tube 1160 may be coupled to the port 1132 at one end. A valve member 1180 may be coupled to the extension tube 1160 at an opposite end such that the valve member 1180 is in fluid communication with the suction chamber 1121 through the extension tube 1160. The position of the valve member 1180 at the end of the extension tube 1160 allows the valve member to be accessed by a syringe or other medical device without breaking the seal formed between the housing 1120 and the patient's skin which may result in a loss of the suction force. The valve member 1180 may be configured to retain a negative gauge pressure or suction force within the suction chamber 1121. In the illustrated embodiment of FIGS. 13-13D, the valve member 1180 is a check-valve 1181. The check-valve 1181 may be selectively opened to allow a negative gauge pressure or suction force to be formed in the suction chamber 1121 and selectively closed to retain the suction force in the suction chamber 1121.

The housing 1120 includes a horizontally oriented post or hook 1135 disposed at an end of the housing 1120 opposite the attachment location of the securement band 1110. In one embodiment, the hook 1135 may be integrally formed with the housing 1120 and formed from the same materials of the housing 1120. In another embodiment, the hook 1135 may be formed as a separate component from a different material and coupled to the housing 1120 using any suitable technique, such as over molding, gluing, bonding, etc. For example, the hook 1135 may be formed from a rigid material while the housing 1120 may be formed from a compliant material. The hook 1135 may include a bar 1139 extending in a horizontal orientation parallel to the longitudinal axis of the flange 1124 and from one side of the housing 1120. A cantilevered or free end 1136 of the bar 1139 may include a flange having a larger width dimension than the bar 1139. A length of the bar 1139 may be sized to accommodate a width of the securement band 1110. In other embodiments, the bar 1139 could be part of a buckle. The bar 1139 can be disposed a distance from the wall 1122 of the housing 1120 such that a space 1137 extends from the free end 1136 to an opposite end of the bar 1139. The space 1137 is sized such that the securement band 1110 can be freely passed through the space 1137. The hook 1135 may permit a clinician to more securely dispose the housing 1120 on the patient's limb which may result in maintenance of a negative gauge pressure or suction force within the suction chamber 1121.

As shown in FIGS. 13-13D, the housing 1120 may be coupled to a securement band 1110. The securement band 1110 may be configured to be disposed around a portion of a patient's limb in order to secure the housing 1120 over an arteriotomy site. For example, the securement band 1110 may be a wrist band configured to be disposed around a wrist of a patient such that the housing 1120 can be secured over a radial or ulnar artery arteriotomy site. In other embodiments, the securement band 1110 may be configured to be disposed around a patient's hand, thigh, ankle, upper arm, etc. The securement band 1110 may be formed of a flexible polymeric film and include a fixation member 1112 and a fastener 1113 disposed at a free end on the securement band 1110. The fixation member 1112 and the fastener 1113 are configured to selectively couple the free end of the securement band 1110 with a portion of the securement band 1110 when disposed around the portion of the patient's limb and around the hook 1135. For example, the fixation member 1112 and the fastener 1113 may be a hook and loop material, snap, button, adhesive, etc. An end of the securement band 1110 opposite the free end may be fixedly coupled to the housing 1120 using any suitable technique, such as gluing, welding, bonding, etc.

In use, as depicted in FIG. 13D, following an arterial catheterization procedure and prior to removal of an introducer sheath, the arteriotomy closure device 1100 may be positioned on a portion of a limb of a patient such that the housing 1120 is disposed over an arteriotomy site by locating the introducer sheath 1150 within the window 1128 of the flange 1124. In certain embodiments, the housing 1120 may be disposed over the arteriotomy site, a skin puncture site, and/or a skin tract between the arteriotomy site and the skin puncture site. The window 1128 may be utilized to position the housing 1120 proximal to the skin puncture site. The securement band 1110 may be wrapped around the portion of the limb in a first direction. The free end of the securement band 1110 may be passed around the bar 1139 of the hook 1135 from a lower side to an upper side and then wrapped partially around the portion of the limb in an opposite direction from the first direction thereby cinching the securement band 1110 and securing the distal sealing surface 1123 to the patient's skin. In another embodiment, a loop or buckle may be formed with the securement band 1110 and the loop passed over the free end of the hook 1135 The securement band 1110 may be cinched securely and the fastener 1113 coupled to the fixation member 1112 such that the housing 1120 may be held tightly to the patient's skin such that the distal sealing surface 1123 forms an airtight seal between the housing 1120 and the patient's skin. In some embodiments, a liquid, gel, or skin protective film may be applied to the patient's skin prior to placement of the housing 1020 to facilitate achieving the airtight seal.

A syringe may be coupled to the check-valve 1181. A plunger of the syringe can be displaced from a distal position to a proximal position to generate a negative gauge pressure or suction force within the syringe and the suction chamber 1121. The plunger may be locked in the proximal position by an optional plunger retention member. The suction force within the suction chamber 1121 may cause the patient's skin, subcutaneous tissue, and artery to be drawn, or distended, in a proximal, or puckered, position toward the housing 1120 and anvil 1170, such that the anvil 1170 applies a counter force against the patient's skin overlying the arteriotomy and/or the tissue tract which facilitates hemostasis, or cessation of bleeding, from the arteriotomy. In certain embodiments, the suction force is formed in the suction chamber 1121 prior to removal of the introducer sheath from the artery. In other embodiments, the introducer sheath is removed from the artery following forming of the suction force in the suction chamber 1121.

When cessation of bleeding from the skin puncture site has been achieved, the plunger may be moved to the distal position such that the suction force in the suction chamber 1121 is relieved and atmospheric pressure inside the suction chamber 1121 is restored, thus removing the counter force provided by the anvil 1170 against the patient's skin. At this time, the arteriotomy closure device 1100 may be removed from the patient's limb.

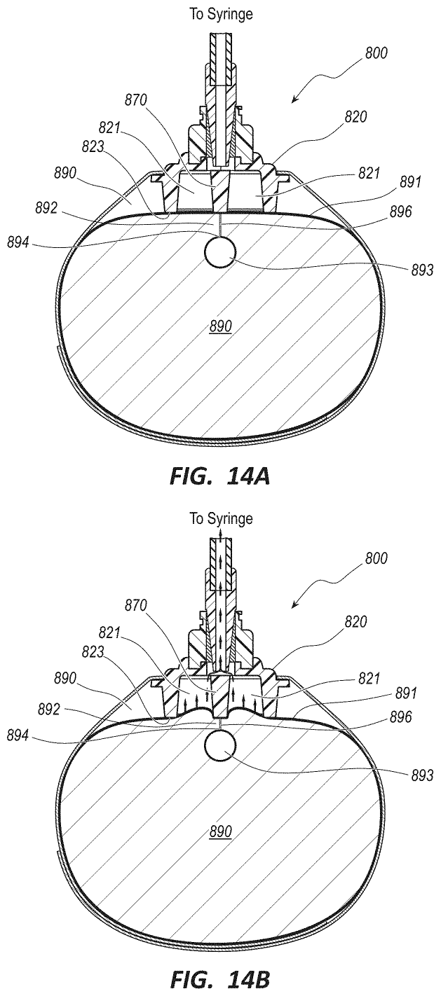

FIG. 14A illustrates a transverse cross-sectional view of an arteriotomy closure device 800, similar to the arteriotomy closure device 300 previously described, coupled to a wrist 890 of a patient in a pre-suction state. The arteriotomy closure device 800 includes a housing 820, a suction chamber 821 and a counterforce member or anvil 870. The housing 820 is disposed over a blood vessel 893 (e.g., artery) with a distal sealing surface 823 in contact with a skin surface 891. The counterforce member 870 is positioned in axial alignment over the blood vessel 893. The blood vessel 893 includes an arteriotomy 894 through a wall of the blood vessel. A tissue tract 896 extends between the arteriotomy 894 and a skin puncture site at the skin surface 890 such that the arteriotomy 894 is in fluid communication with the skin puncture surface 891. The housing 820 may be positioned on the wrist 890 such that the skin puncture site is disposed exterior to the suction chamber 821. In other embodiments, the skin puncture site may be disposed within the suction chamber 821 and beneath the anvil 870.

FIG. 14B illustrates a longitudinal cross-sectional view of the arteriotomy closure device 800 coupled to the wrist 890 in a suction state. The suction chamber 821 contains a negative gauge pressure or suction force. The amount of fluid displaced to achieve a desired negative gauge pressure may be, in some embodiments, between 5-30 mL. In other embodiments, the amount of fluid displaced to achieve a desired negative gauge pressure is between 5-10 mL. The distal sealing surface 823 forms an airtight seal to the skin surface 890. The skin surface 890 within the perimeter of the distal sealing surface 823 is drawn or puckered into the suction chamber 821 causing subcutaneous tissue 892 and the blood vessel 893 to be drawn upward. The skin surface 890 is drawn against the counterforce member 820 causing the subcutaneous tissue 892 to be compressed. Compression of the subcutaneous tissue 892 causes closure of the tissue tract 896 and the arteriotomy 894 while not applying a distorting compressive force to the blood vessel 893. Closure of the tissue tract 896 and the arteriotomy 894 may result in cessation of blood flow from the skin surface 890. In some embodiments, it may take between 15 minutes to 6 hours of the application of negative gauge pressure to achieve and maintain hemostasis. In other embodiments, it may take between 2 to 3 hours of the application of negative gauge pressure to the treatment site to achieve and maintain hemostasis.

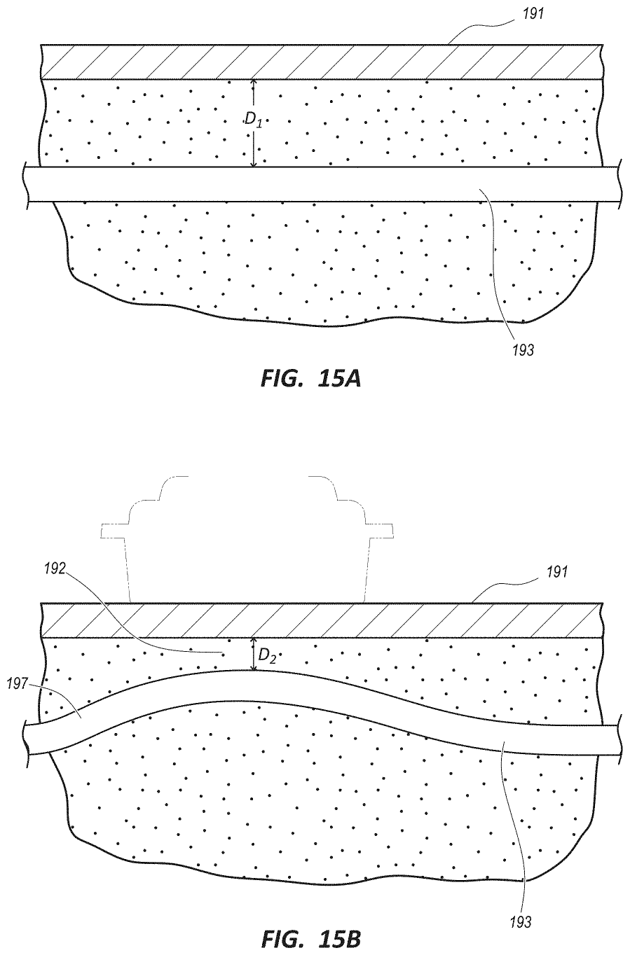

FIG. 15A is an illustration of a sonogram image produced from an ultrasound examination of a patient's right radial artery 193 in a lateral, longitudinal orientation. The artery 193 is shown in a natural state whereby the arterial lumen 197 is fully perfused with blood, prior to suction being applied to the skin surface 191 by any arteriotomy closure devices disclosed herein. It can be seen from the illustration of FIG. 15A that the longitudinal orientation of the artery 193 is substantially parallel with the patient's skin surface 191. The artery 193 is also shown at a depth of D₁ below the skin surface 191.

FIG. 15B is an illustration of a sonogram image produced from an ultrasound examination of the right radial artery 193 in a lateral, longitudinal orientation. The artery 193 is shown in a state where suction has been applied to the skin surface 191 directly overlying the artery 193. It can be seen from the illustration of FIG. 15B that the longitudinal orientation of the artery 193 has been substantially altered by the application of suction such that the artery 193 is bowed in an upward direction, i.e., the suction has reoriented the artery 193 from being substantially parallel with the skin surface 191 to being bow-shaped. It can further be seen that the depth D₂ of the artery 193 is less than the depth D₁ due to compression of subcutaneous tissue 192. It can additionally be seen from the illustration that the arterial lumen 197 remains fully perfused with blood during the application of suction to the skin surface 191 directly overlying the artery 193.

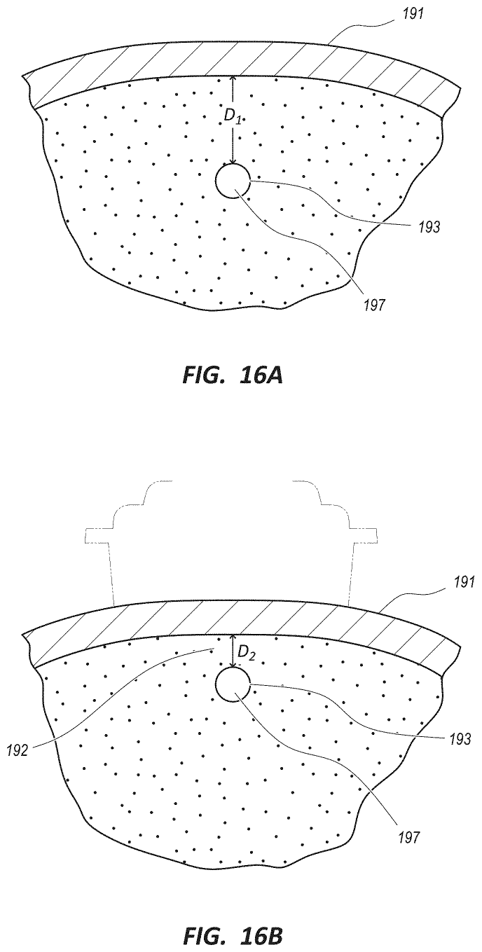

FIG. 16A is an illustration of a sonogram image produced from an ultrasound examination of the right radial artery 193 in a transverse orientation (i.e., a cross-sectional view). The artery 193 is shown in a natural state whereby the arterial lumen 197 is fully perfused with blood, prior to suction being applied to the skin surface 191. It can be seen from the illustration that the cross-sectional geometry of the arterial lumen 197 is substantially round and the artery 193 is at a depth D₁ below the skin surface 191.

FIG. 16B is an illustration of a sonogram image produced from an ultrasound examination of the right radial artery 193 in a transverse orientation (i.e., a cross-sectional view). The artery 193 is shown in a state where suction has been applied to the skin surface 191 directly overlying the artery 193. It can be seen from the illustration that the subcutaneous tissue 192 directly overlying the artery 193 has been substantially compressed and the depth D₂ of the artery 193 is less than the depth D₁. It can further be seen from the image of the arterial lumen 197 that the cross-sectional geometry of the arterial lumen 197 remains substantially round under the application of suction, i.e., the application of suction to the skin surface 191 directly overlying the artery 193 has had no effect on the artery's perfusion state, nor has the artery 193 been flattened, or compressed, to be altered in shape from its natural state.

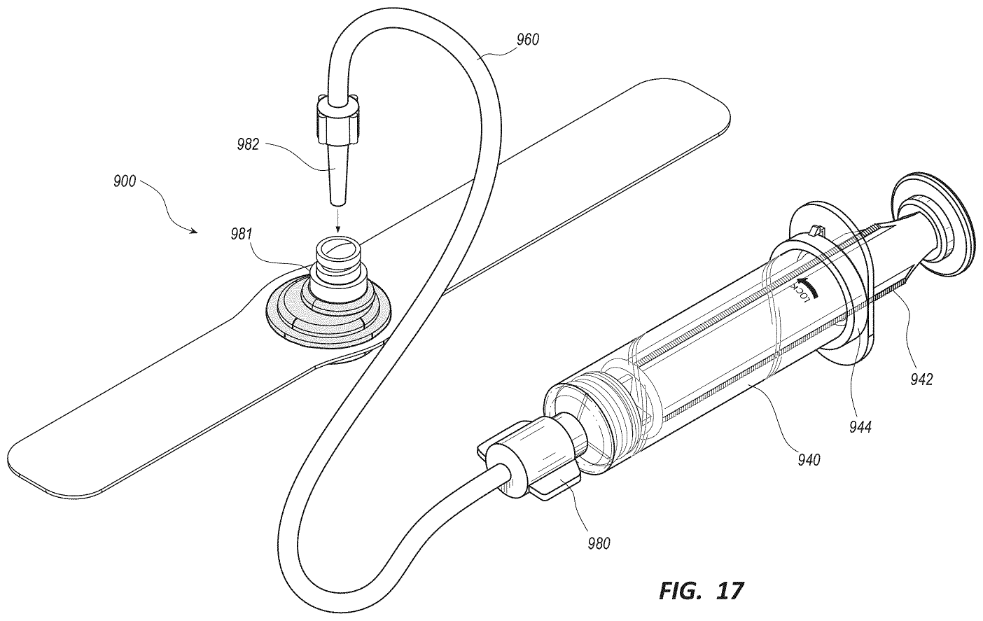

FIG. 17 depicts a kit or system that may be used to provide hemostasis at an arteriotomy following a vascular access procedure. As depicted in FIG. 14, the system or kit may include an arteriotomy closure device 900, an extension tubing member 960, and a suction generating member (e.g., syringe) 940. The arteriotomy closure device 900 may be any one of the previously described embodiments of an arteriotomy closure device. The extension tubing member 960 may include a distal fitting 982 having a distally extending protuberance configured to access a check-valve 981 of the arteriotomy closure device 900. The distal fitting 982 may be configured to access the check-valve 981 using a straight distal displacement such that a lateral force is not applied to the check-valve 981 causing the arteriotomy closure device 900 to break an airtight seal at the skin surface of the patient. The extension tubing member 960 may include a valve member 980 coupled to a proximal end. In other embodiments, the extension tubing member 960 may include a female luer fitting coupled to the proximal end. The syringe 940 is configured to couple with the proximal end of the extension tubing member 960 such that the syringe 940 is in fluid communication with the arteriotomy closure device 900 when the check-valve 981 is accessed by the distal fitting 982. A negative gauge pressure or suction force may be formed within the syringe 940 when a plunger 942 is displaced proximally. The plunger 942 may be maintained proximally displaced by an optional plunger locking member 944.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. An arteriotomy closure device, comprising:
a housing defining a suction chamber; and
an anvil disposed within the suction chamber.

2. The arteriotomy closure device of claim 1, wherein the suction chamber is in fluid communication with a fluid displacement member configured to induce a negative gauge pressure within the suction chamber.

3. The arteriotomy closure device of claim 1, wherein the housing comprises a distal sealing surface configured to form an airtight seal against a patient's skin.

4. The arteriotomy closure device of claim 1, further comprising at least one window configured to permit a user to visualize a skin puncture site during placement of the arteriotomy closure device.

5. The arteriotomy closure device of claim 1, wherein the closure device comprises a channel configured to receive a portion of an introducer sheath while maintaining a negative gauge pressure within the suction chamber.

6. The arteriotomy closure device of claim 1, further comprising a flange extending from the housing and configured to abut tissue adjacent an arteriotomy site.

7. The arteriotomy closure device of claim 1, further comprising a suction retention member in fluid communication with the suction chamber and configured to selectively retain a negative gauge pressure within the suction chamber.

8. The arteriotomy closure device of claim 1, wherein in the anvil extends distally into the suction chamber and is configured to abut tissue over an arteriotomy when negative gauge pressure is induced in the suction chamber.

9. The arteriotomy closure device of claim 1, wherein the anvil comprises a distal surface configured to apply pressure to a patient's skin, subcutaneous tissue, and artery proximally against the distal surface when negative gauge pressure is induced in the suction chamber.

10. The arteriotomy closure device of claim 9, wherein the distal surface of the anvil is recessed within the housing from a distal sealing surface of the housing.

11. The arteriotomy closure device of claim 1, further comprising a securement band configured to secure the housing to a limb of a patient such that the suction chamber is positioned adjacent to the radial artery.

12. A method for achieving hemostasis at an access site of an artery, comprising:
providing an arteriotomy closure device, the device comprising:
a housing defining a suction chamber; and
an anvil disposed within the suction chamber;
securing the arteriotomy closure device to a patient such that the suction chamber is positioned adjacent to the arteriotomy; and
inducing a negative gauge pressure within the suction chamber;
wherein a patient's skin, subcutaneous tissue, and artery are drawn proximally against the anvil and an arteriotomy is closed.

13. The method of claim 12, wherein inducing the negative gauge pressure within the suction chamber comprises proximally displacing a syringe plunger.

14. The method of claim 12, further comprising withdrawing an introducer sheath from the artery prior to inducing a negative gauge pressure within the suction chamber.

15. The method of claim 12, further comprising withdrawing an introducer sheath from the artery following inducing a negative gauge pressure within the suction chamber.

16. The method of claim 12, wherein securing the arteriotomy closure device to a limb of the patient comprises coupling the securement band to a hook of the arteriotomy closure device.

17. A system to provide hemostasis at an access site of an artery, comprising:
an arteriotomy closure device, comprising;
a housing defining a suction chamber; and
an anvil disposed within the suction chamber; and
a suction force generating member, comprising;
a barrel; and
a plunger.

18. The system of claim 17, wherein the arteriotomy closure device further comprises a securement band.

19. The system of claim 17, further comprising a tubing that fluidly interconnects the housing and the suction force generating member.

20. The system of claim 19, further comprising a suction force retention member in communication with the suction chamber, the tubing, and the suction force generating member.
